(19) United States
(12) Patent Application Publication
(10) Pub. No.: US 2015/0050694 A1
(43) Pub. Date: Feb. 19, 2015

(54) METHOD OF STERILIZING SEPARATION MEMBRANE MODULE, METHOD OF PRODUCING CHEMICAL BY CONTINUOUS FERMENTATION, AND MEMBRANE SEPARATION-TYPE CONTINUOUS FERMENTATION APPARATUS

(71) Applicant: TORAY INDUSTRIES, INC., Tokyo (JP)

(72) Inventors: Norihiro Takeuchi, Otsu-shi (JP); Atsushi Kobayashi, Otsu-shi (JP)

(73) Assignee: TORAY INDUSTRIES, INC., Tokyo (JP)

(21) Appl. No.: 14/385,627

(22) PCT Filed: Mar. 1, 2013
(86) PCT No.: PCT/JP2013/055711

§ 371 Date: Sep. 16, 2014

(30) Foreign Application Priority Data
Mar. 16, 2012 (JP) 2012-060694

(51) Int. Cl. C12M 1/12; B01D 69/08; B01D 65/02

(52) U.S. Cl. CPC C12M 37/02; B01D 65/022; B01D 69/08; B01D 2321/08
USPC 435/88; 210/636; 435/139; 435/297.1; 435/155; 435/106; 435/161; 435/158; 435/159; 435/143; 435/145; 435/144; 435/87

(57) ABSTRACT

A method of sterilizing a separation membrane module using water vapor includes: a liquid supplying step of supplying a liquid having a boiling point of 80° C. or higher at atmospheric pressure to a secondary side of the separation membrane module such that a filling ratio of the liquid in a space surrounded by a filtration portion of a separation membrane is 70% or more, the filtration portion being used for filtration; a liquid sealing step of isolating the secondary side of the separation membrane module such that the filling ratio of the liquid supplied to the secondary side in the liquid supplying step is 70% or more; and a sterilization step of sterilizing the separation membrane module by supplying water vapor to a primary side of the separation membrane module while the secondary side of the separation membrane module is isolated.

### FIELD

The present invention relates to a method of sterilizing a separation membrane module that is used, for example, to filter microorganisms from, for example, a fermented liquid in order to obtain a chemical contained in the fermented liquid, to a method of producing a chemical by continuous fermentation, and to a membrane separation-type continuous fermentation apparatus.

### BACKGROUND

Fermentation methods, i.e., material production methods involving cultivation of microorganisms or cultured cells can be broadly classified into (1) a batch fermentation method and a fed-batch fermentation method and (2) a continuous fermentation method.

The batch fermentation method and the fed-batch fermentation method in (1) above have advantages in that their facilities are simple, that cultivation is finished in a short time, and that damage due to contamination with germs is small. However, the concentration of the chemical in the fermentation culture solution increases with time, and therefore the productivity and yield of the chemical decrease because of the influence of osmotic pressure, inhibition by the chemical, etc. Therefore, it is difficult to stably maintain high yield and high productivity for a long time.

The continuous fermentation method in (2) above is characterized in that high yield and high productivity can be maintained for a long time because accumulation of the target chemical at a high concentration in a fermenter is avoided. As for the continuous fermentation method, a continuous cultivation method for fermentation of L-glutamic acid or L-lysine has been disclosed (see Non Patent Literature 1). However, in this example, a feedstock is continuously supplied to a fermentation culture solution, and at the same time the fermentation culture solution containing a microorganism or cultured cells is drawn out. Therefore, the microorganism or cultured cells in the fermentation culture solution are diluted, so that improvement in production efficiency is limited.

In one proposed continuous fermentation method, a microorganism or cultured cells is filtered using separation membranes to collect a chemical from the filtrate, and at the same time the microorganism or cultured cells in a retentate are held in the fermentation culture solution or refluxed to thereby maintain the concentration of the microorganism or cultured cells in the fermentation culture solution at a high level. For example, in one proposed technique, continuous fermentation is performed in a continuous fermentation apparatus that uses, as separation membranes, flat membranes formed of an organic polymer (see Patent Literature 1).

In such continuous fermentation, it is preferable to cultivate a pure culture with contamination with germs prevented. When germs are introduced from, for example, the separation membrane module during filtration of the fermentation culture solution, the chemical cannot be efficiently produced because of a reduction in fermentation efficiency, foaming in the fermenter, etc. Therefore, the fermenter, its peripheral facilities, and the separation membranes must be sterilized before fermentation in order to prevent contamination with germs.

Examples of the sterilization method may include flame sterilization, dry heat sterilization, boiling sterilization, steam sterilization, ultraviolet ray sterilization, gamma ray sterilization, and gas sterilization. However, it should be noted that when moisture in the pores of the separation membranes is lost and the separation membranes are dried, its separation function disappears. A sterilization method using an agent may be used. However, this method has a problem with post treatment of the agent after sterilization and a problem with the agent remaining in the separation membrane module. In addition, it is feared that a microorganism with resistance to the agent may remain.

The separation membranes may be membranes with a flat shape, membranes with a hollow fiber shape, membranes with a spiral form, etc. When a hollow fiber membrane module is used, examples thereof may include the external pressure type and the inner pressure type. Particularly, the hollow fiber membrane module has a large membrane area per single unit and therefore has an industrially useful structure, but the structure is complicated.

To sterilize separation membranes with a complicated structure without drying, steam sterilization (generally at 121° C. for 15 minutes to 20 minutes) is suitable. In the steam sterilization, water vapor at a prescribed temperature is supplied to the separation membrane module to sterilize it.

Generally, when the steam sterilization is performed in a production scale facility such as a fermenter, steam at a prescribed temperature and a prescribed pressure, for example, saturated water vapor at 125° C., is supplied to the fermenter and its peripheral facilities to increase the temperature of the facilities to 121° C., which is a general steam sterilization temperature. Then the sterilization temperature is maintained for a prescribed time (at least 20 minutes) to perform steam sterilization.

In one proposed steam sterilization method, water vapor is introduced to the outer side (primary side) of hollow fiber membranes during steam sterilization or to the outer side and also the inner side (secondary side) of the hollow fiber membranes to subject them to steam sterilization (Patent Literature 2). In Patent Literature 2, a trial test of the steam sterilization method during long-term operation of a hollow fiber membrane module was performed by repeating injection of water into the hollow fiber membrane module and injection of water vapor thereinto to evaluate leakage. However, steam sterilization was not performed with water sealed on the secondary side of the separation membrane module.

When the apparatus is cooled directly after steam sterilization, the water vapor is condensed, and a negative pressure is generated in the apparatus, causing the fear of contamination with germs.

In one technique proposed to address the above issue, after steam sterilization of a semipermeable membrane module, hot water is introduced from a raw water side to prevent a negative pressure in a filtration apparatus from being generated (see Patent Literature 3). In another proposed technique, after steam sterilization of a membrane module, raw water at room temperature is introduced at a linear velocity lower than that during filtration treatment to cool the module (see Patent Literature 4).

In another proposed method for steam sterilization of a hollow fiber membrane module, after steam sterilization, air is supplied to the inner side, i.e., the raw solution side (primary side), of hollow fiber membranes, and part of the air is allowed to pass through the hollow fiber membranes toward their outer side, i.e., their transmission side (secondary side), to fill the space on the transmission side. Then water is supplied from the raw solution side to reduce the temperature of the module (see Patent Literature 5).

### CITATION LIST

### Patent Literature

Patent Literature 1: Japanese Laid-open Patent Publication No. 2007-252367
Patent Literature 2: Japanese Laid-open Patent Publication No. 2-207826
Patent Literature 3: Japanese Laid-open Patent Publication No. 61-242605
Patent Literature 4: Japanese Laid-open Patent Publication No. 8-164328
Patent Literature 5: Japanese Examined Patent Application Publication 8-4726

### Non Patent Literature

Non Patent Literature 1: Toshihiko Hirao et al., Appl. Microbiol. Biotechnol., 32, 269-273 (1989)

### SUMMARY

### Technical Problem

When a fermenter, its peripheral piping, etc. are subjected to steam sterilization, the temperature of the supplied water vapor is set such that the temperature in a place (cold spot) in which its temperature is most difficult to increase is equal to or higher than a prescribed steam sterilization temperature. The supply time of water vapor is set such that, after the temperature of the place in which its temperature is most difficult to increase is increased to a prescribed steam sterilization temperature or higher, sterilization is performed for a prescribed steam sterilization time or longer. Generally, the temperature of the supplied water vapor is set to 121° C. or higher with heat dissipation measures such as thermal insulation taken.

However, when the temperature of the supplied water vapor is high and the supply time is long, it is feared that the components of the separation membrane module may deteriorate because they come into contact with the high-temperature water vapor for a long time. For example, in a hollow fiber membrane module, a urethane- or epoxy-based potting agent is generally used to secure hollow fiber membranes to a module container. However, it is feared that this potting agent may be degraded by repeatedly performed steam sterilization and therefore may peel off the hollow fiber membranes or the module container. In the hollow fiber membrane module, a highly stretchable urethane-based resin is used as the potting agent for some cases. However, deterioration of the urethane resin proceeds when temperature exceeds 120° C. Therefore, when the potting agent comes into contact with water vapor at 121° C. or higher, which is a general steam sterilization treatment temperature, for a long time, it is feared that the potting agent may deteriorate and leakage may occur.

Water vapor tends to flow into a space with small pressure loss. Therefore, it is feared that water vapor may be less likely to flow into a portion in which the density of separation membranes is excessively high, for example, a portion in which hollow fiber membranes are excessively densely packed. During steam sterilization, the separation membranes are held at high temperature and saturated water vapor pressure. In the portion in which the density of separation membranes is excessively high, their temperature is increased mainly by heat transfer, so that a long time is required to increase the temperature to the steam sterilization conditions. Separation membranes in a dense shape have an advantage in that a large membrane area can be obtained. However, when the density is excessively high, water vapor is not sufficiently distributed during steam sterilization, and this causes a problem in that sterilization failure occurs because temperature is not increased to the sterilization temperature or a problem in that a long time is required to increase the temperature to reliably perform sterilization.

When steam sterilization is performed for a long time, moisture in the pores of the separation membranes comes into contact with saturated water vapor during steam sterilization, is equilibrated with the saturated water vapor, and gradually reduced in amount. In this case, it is feared that the degree of drying of the separation membranes may increase. When the separation membrane module is left to cool, the temperature inside the separation membrane module is not uniform in many cases, and it is feared that the separation membranes may be dried when they come into contact with high-temperature components such as the casing of the separation membrane module. When moisture in hollow fiber membranes is vaporized, the vapor phase in the pores of the separation membranes must be replaced with a liquid phase in order to perform filtration treatment later. Hydrophilic separation membranes are wettable with water, and therefore replacement is easy. However, separation membranes having the required performance such as chemical resistance and heat resistance are often formed from hydrophobic materials as base materials. To replace the vapor phase in the pores of such separation membranes with a liquid phase, the vapor phase must be first replaced with, for example, a liquid having an affinity for the hydrophobic membranes and then replaced with water.

The present invention has been made in view of the above circumstances and provides a separation membrane module sterilization method that can reliably sterilize the separation membrane module in a short time with drying of the separation membranes suppressed. The present invention also provides a method of producing a chemical by continuous fermentation and a membrane separation-type continuous fermentation apparatus.

### Advantageous Effects of Invention

To solve the above-described problem and achieve the object, a method of sterilizing a separation membrane module according to the present invention uses water vapor and includes: a liquid supplying step of supplying a liquid having a boiling point of 80° C. or higher at atmospheric pressure to a secondary side of the separation membrane module such that a filling ratio of the liquid in a space surrounded by a filtration portion of a separation membrane is 70% or more, the filtration portion being used for filtration; a liquid isolating step of isolating the secondary side of the separation membrane module such that the filling ratio of the liquid supplied to the secondary side in the liquid supplying step is 70% or more; and a sterilization step of sterilizing the separation membrane module by supplying water vapor to a primary side of the separation membrane module while the secondary side of the separation membrane module is isolated.

Moreover, a method of producing a chemical by continuous fermentation according to the present invention includes: a steam sterilization step of using the above-described method of sterilizing to sterilize the separation membrane module; a fermentation step of converting a fermentation feedstock to a fermented liquid containing a chemical by fermentation culture by a microorganism; and a membrane separation step of collecting the chemical as a filtrate from the fermented liquid using the separation membrane module subjected to the steam sterilization step.

Moreover, a membrane separation-type continuous fermentation apparatus according to the present invention includes: a fermenter configured to convert a fermentation feedstock to a fermented liquid containing a chemical by fermentation cultivation of the fermentation feedstock using a microorganism; a separation membrane module configured to separate the chemical from the fermented liquid; a fermented liquid circulation unit configured to feed the fermented liquid from the fermenter to the separation membrane module; a steam supply unit configured to supply water vapor to the fermenter and the separation membrane module; a liquid supply unit configured to supply a liquid having a boiling point of 80° C. or higher at atmospheric pressure to a secondary side of the separation membrane module; and an isolation unit configured to isolate the secondary side of the separation membrane module such that a filling ratio of the liquid in a space surrounded by a filtration portion of a separation membrane is 70% or more during operation of the stream supply means, the filtration portion being on the secondary side of the separation membrane module and used for filtration.

In the present invention, the liquid at 80° C. or higher is sealed on the secondary side of the separation membrane module at atmospheric pressure, and then water vapor is supplied to the primary side. In this manner, the time required for the separation membrane module to be heated to a prescribed sterilization temperature can be significantly reduced. Therefore, the thermal deterioration of the potting agent etc. can be suppressed, and the drying of the separation membrane can also be suppressed. In addition, since air is not used for cooling etc., breakage of the separation membrane and a reduction in the amount of water permeation can be suppressed.

### BRIEF DESCRIPTION OF DRAWINGS

FIG. 1 is a schematic diagram of a separation membrane module sterilizing apparatus according to a first embodiment of the present invention.

FIG. 2 is a flowchart explaining steam sterilization treatment according to the first embodiment of the present invention.

FIG. 3 is a schematic diagram of a separation membrane module sterilizing apparatus according to a first modification of the first embodiment of the present invention.

FIG. 4 is a schematic diagram of a separation membrane module sterilizing apparatus according to a second modification of the first embodiment of the present invention.

FIG. 5 is a schematic diagram of a membrane separation-type continuous fermentation apparatus according to a second embodiment of the present invention.

FIG. 6 is a flowchart explaining sterilization treatment according to the second embodiment of the present invention.

FIG. 7 is a schematic diagram of a membrane separation-type continuous fermentation apparatus according to a first modification of the second embodiment of the present invention.

### DESCRIPTION OF EMBODIMENTS

A method of sterilizing a separation membrane module, a method of producing a chemical by continuous fermentation, and a membrane separation-type continuous fermentation apparatus according to embodiments of the present invention will next be described with reference to the drawings. However, the present invention is not limited to the embodiments described below.

### First Embodiment

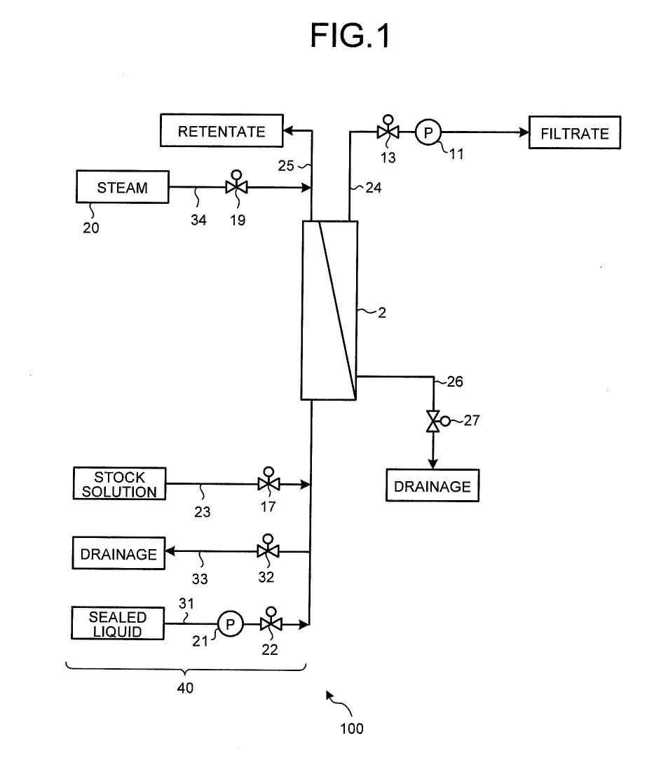

A separation membrane module sterilization method according to a first embodiment of the present invention will be described with reference to FIG. 1. FIG. 1 is a schematic diagram of a separation membrane module sterilizing apparatus according to the first embodiment of the present invention. A sterilizing apparatus 100 includes: a vapor supply unit 20 for supplying water vapor to the primary side of a separation membrane module 2; and a liquid supply unit 40 for supplying a liquid with a boiling point of 80° C. or higher at atmospheric pressure to the secondary side of the separation membrane module 2. A circulation valve 17 and a pipe 23 for supplying a stock solution to be treated are connected to the primary side of the separation membrane module 2, and a filtrate discharge line 24 for discharging the filtrate filtered through separation membranes to the outside of the separation membrane module 2 is connected to the secondary side of the separation membrane module 2. A filtration pump 11 and a filtration valve 13 are provided in the filtrate discharge line 24. The stock solution is filtered from the primary side to the secondary side when the filtration valve 13 is opened to suck the stock solution by the filtration pump 11. The stock solution not filtered to the secondary side is crossflow filtered via a pipe 25.

The vapor supply unit 20 is connected to the primary side of the separation membrane module 2 through a supply valve 19 and a pipe 34. The water vapor at a prescribed temperature supplied from the vapor supply unit 20 to the primary side of the separation membrane module 2 is discharged to the outside of the separation membrane module 2 through a discharge line 33 and a discharge valve 32. The liquid supplied from the liquid supply unit 40 to the primary side of the separation membrane module 2 is supplied to the secondary side through the separation membranes. It is preferable that the liquid be filtered to the secondary side while sucked by the filtration pump 11. The liquid supplied to the primary side of the separation membrane module 2 is discharged to the outside of the separation membrane module 2 through the pipe 25 for the crossflow filtration. Hereinafter, the side of the separation membrane module 2 on which the separation membrane module 2 comes into contact with the stock solution to be treated is referred to as the primary side, and the side on which the separation membrane module 2 comes into contact with the treated filtrate is referred to as the secondary side.

The separation membrane module 2 includes separation membranes and a container for accommodating the separation membranes. The separation membranes used in the first embodiment are any of organic and inorganic membranes. When the separation membranes are washed, backwashing or washing by immersion in a chemical solution is performed. Therefore, preferably, the separation membranes are durable against these washing processes. The separation membranes used may be any of membranes with a flat shape, membranes with a hollow fiber shape, membranes with a spiral form, etc. Particularly, a hollow fiber membrane module is preferred. The hollow fiber membrane module used may be any of the external pressure type and the inner pressure type.

From the viewpoint of separation performance, water permeability, and also fouling resistance, an organic macromolecular compound can be suitably used for the separation membranes used in the first embodiment. Examples of the organic macromolecular compound may include polyethylene-based resins, polypropylene-based resins, polyvinyl chloride-based resins, polyvinylidene fluoride-based resins, polysulfone-based resins, polyethersulfone-based resins, polyacrylonitrile-based resins, cellulose-based resins, and cellulose triacetate-based resins. The organic macromolecular compound may be a mixture including any of the above resins as a main component.

Polyvinyl chloride-based resins, polyvinylidene fluoride-based resins, polysulfone-based resins, polyethersulfone-based resins, and polyacrylonitrile-based resins are preferably used because they are easily formed into a membrane using a solution and have good physical durability and chemical resistance. A polyvinylidene fluoride-based resin or a resin containing the polyvinylidene fluoride-based resin as a main component is more preferably used because of their characteristics of having both chemical strength (particularly chemical resistance) and physical strength.

The polyvinylidene fluoride-based resin used is preferably a homopolymer of vinylidene fluoride. The polyvinylidene fluoride-based resin used may be a copolymer of vinylidene fluoride and a vinyl-based monomer copolymerizable therewith. Examples of the vinyl-based monomer copolymerizable with vinylidene fluoride may include tetrafluoroethylene, hexafluoropropylene, and trichlorofluoroethylene.

The average pore diameter of the separation membranes used in the first embodiment may be appropriately determined according to the intended use and situation. The average pore diameter is preferably small to some extent and is generally preferably 0.01 μm or more and 1 μm or less. If the average pore diameter of the hollow fiber membranes is less than 0.01 μm, the pores are clogged with membrane fouling components such as sugar and protein components and aggregates thereof, so that stable operation cannot be performed. In consideration of the balance with water permeability, the average pore diameter is preferably 0.02 μm or more and more preferably 0.03 μm or more. If the average pore diameter exceeds 1 μm, fouling components are not sufficiently separated from the pores by shear force caused by the smoothness of the membrane surface and a flow on the membrane face and by physical washing such as backwashing and air scrubbing, so that stable operation cannot be performed.

When the average pore diameter is close to the size of a microorganism or cultured cells, the pores may be clogged directly with the microorganism or cultured cells. In addition, cell debris may be produced when part of the microorganism or cultured cells in the fermented liquid die. To prevent the pores from being clogged with the cell debris, the average pore diameter is preferably 0.4 μm or less and more preferably 0.2 μm or less.

The average pore diameter of the separation membranes can be determined by measuring diameters of a plurality of pores observed under a scanning electron microscope at a magnification of 10,000× or higher and then averaging the measured diameters. Preferably, the average pore diameter is determined by randomly selecting ten or more particles, preferably twenty or more particles, measuring the diameters of the selected pores, and then computing the number average of the measured diameters. When the pores are, for example, not circular, the following method can be used preferably. Circles having the same areas as those of the pores, i.e., equivalent circles, are determined using, for example, an image processing device, and the diameters of the equivalent circles are used as the diameters of the pores.

When filtration treatment using the separation membrane module 2 is performed, it is preferable to subject the separation membrane module 2 to steam sterilization treatment before the filtration treatment, in order to prevent contamination of the inside of the apparatus and/or the filtrate with germs etc.

In the first embodiment, it is preferable that the liquid from the liquid supply unit 40 be sealed on the secondary side of the separation membrane module 2 before water vapor is supplied from the vapor supply unit 20 to the primary side of the separation membrane module 2 and further that the water vapor be supplied from the vapor supply unit 20 with the liquid being sealed.

When a liquid having a high boiling point, for example, a liquid having a boiling point of 80° C. or higher at atmospheric pressure, is sealed on the secondary side of the separation membrane module 2 before steam sterilization is performed, the sealed liquid transfers heat from the water vapor supplied to the primary side of the separation membrane module 2 to each part of the separation membrane module 2 through the separation membranes. In this manner, the time required to increase the temperature of the separation membrane module 2 to a prescribed sterilization temperature can be shorter than that when no liquid is sealed. Therefore, a heat load on the separation membrane module 2 can be reduced.

The thermal conductivity of a liquid is generally higher than that of a gas (for example, the thermal conductivity of water is higher than the thermal conductivity of air and the thermal conductivity of water vapor). Therefore, by supplying water vapor after a liquid is supplied to the secondary side of the separation membrane module 2 and then the secondary side is isolated, the rate of temperature increase in the separation membrane module 2 is faster than that when air or water vapor is present on the secondary side. The smaller the heat capacity of the liquid to be sealed, the more it is advantageous for increasing the temperature. Therefore, the heat conduction in the separation membrane module 2 may also be influenced by the value of heat capacity.

Particularly, when, for example, the diameter of the pores of the membranes is large or the membranes are formed from a material having affinity to water vapor, water vapor can pass through the separation membranes from the primary side to the secondary side in some cases. Therefore, when the liquid is sealed on the secondary side in advance before heating with water vapor, part of the pressurized water vapor supplied to the primary side passes from the primary side to the secondary side. At the same time, the liquid passes through the separation membranes from the secondary side to the primary side, or the liquid on the secondary side is increased in temperature and vaporized, so that room for introducing water vapor is generated on the secondary side. This allows the liquid on the secondary side to be replaced with the water vapor. The membranes can thereby be heated with the water vapor also from the secondary side.

When steam sterilization is performed for a long time by introducing water vapor with no liquid sealed on the secondary side, moisture in the pores of the separation membranes comes into contact with the saturated water vapor during steam sterilization and is equilibrated with the saturated water vapor. This causes the amount of moisture in the pores of the separation membranes to be reduced gradually, so that it is feared that the separation membranes may be dried. In addition, since water vapor does not always pass through the hollow fiber membranes uniformly, part of air present on the secondary side of the hollow fiber membranes before the introduction of water vapor remains on the secondary side, and it is feared that the air may build up in a locked state (i.e., with air lock formed).

When the secondary side is isolated with no liquid sealed on the secondary side, it is necessary that air on the secondary side be moved to the primary side in order to allow water vapor to move to the secondary side through the separation membranes. However, air cannot pass through the separation membranes unless a pressure higher than a bubble point is applied. The pressure applied to the primary side of the separation membranes during steam sterilization varies depending on the material of the membranes but is often less than the bubble point particularly when the separation membranes are hydrophobic. For example, in the pressure conditions in general steam sterilization, the pressure applied is a saturated water vapor pressure at about 121° C. and is therefore about 0.13 MPa. In this case, air cannot pass through the separation membranes and builds up on the secondary side of the hollow fiber membranes in a locked state (i.e., with air lock formed). Since the primary side is in a pressurized state, the air on the secondary side cannot be transmitted to the secondary side unless the pressure of the air is equal to or higher than the pressure on the primary side. Therefore, it is difficult to heat the hollow fiber membranes from the secondary side unless the liquid is sealed on the secondary side.

In the present invention, the term “sealed” means that a space filled with a liquid is isolated so that the liquid does not flow out of the space. The term “isolated” means that a prescribed space is isolated from the outside space. The phrase “isolated from the outside space” can translate into “separated from the outside space.” Particularly in the separation membrane module, the term “isolated” means that the paths through which the liquid in the space on the secondary side of the separation membranes flows are closed.

Specific means for isolation is, for example, to close valves in paths which are connected to the separation membrane module and through which the liquid on the secondary side of the separation membranes flows. More specifically, the “isolated state” is a state in which the valves 13 and 27 provided in the lines 24 and 26 connected to the separation membrane module 2 are closed so that no liquid passes through the valves. Valves 14 and 22 are also closed if this is required for isolation. However, as described later, the valve 22 is opened when steam sterilization is performed while counter pressure filtration is performed.

As described above, the liquid may pass through the separation membranes, and this depends on the separation membranes and the operating conditions. However, the liquid passing through the separation membranes does not correspond to an “outflow.” Specifically, even when the liquid passes through the separation membranes, this state is included in the “isolated state.”

The terms “sealed” and “isolated” are not meant to absolutely exclude outflows other than the outflow through the separation membranes. Specifically, an outflow of the liquid is not excluded, so long as the effect of improving the sterilization efficiency is achieved by the sealed liquid as described above. A reduction in filling ratio after the start of steam sterilization is permitted.

The state in which counter pressure filtration is performed, i.e., the liquid is supplied to the secondary side and allowed to pass through the separation membranes from the secondary side to the primary side, also corresponds to the “sealed” and “isolated” in the present invention. The details will be described later.

The sterilization temperature of general steam sterilization is 121° C. Therefore, the boiling point of the liquid to be sealed at atmospheric pressure is preferably 80° C. or higher, in order to reduce the influence of vaporization of the sealed liquid on the separation membrane module 2. When sterilization is performed with the sterilization temperature set to be lower than 121° C., a liquid with a boiling point of 80° C. or lower at atmospheric pressure can also be selected as the liquid to be sealed.

For example, water such as ion exchanged water, water filtered through a reverse osmosis membrane, or distilled water or an alcohol is preferably used as the liquid supplied to the secondary side of the separation membrane module 2. Examples of the alcohol may include: monohydric alcohols such as 1-butanol, 2-butanol, and 1-heptanol; polyhydric alcohols such as ethylene glycol, 1,2-propanediol, 1,3-propanediol, diethylene glycol, triethylene glycol, and glycerin; and butyl cellosolve and phenyl cellosolve. Silicone oil and water containing a surfactant added thereto can also be used. In addition, water containing an electrolyte dissolved therein may be used, and water containing an alkali, an acid, an oxidizing agent, or a reducing agent added thereto may be used. However, it is preferable to check in advance that the separation membranes, the module components, etc. are not adversely affected by, for example, a decomposed product generated therefrom. For example, when the sealed liquid remains present, the remaining sealed liquid may be mixed with the filtrate. In consideration of this, water containing no additives is preferred as the liquid to be sealed.

When the liquid to be sealed has a high affinity for the separation membranes, the liquid can be easily sealed on the secondary side of the separation membrane module 2. Therefore, it is preferable that, when the separation membranes are hydrophilic, a hydrophilic liquid be selected, and that, when the separation membranes are hydrophobic, a hydrophobic liquid be selected. Even when the separation membranes used are hydrophobic, a hydrophilic liquid such as water can be selected as the sealed liquid. In this case, the hydrophobic separation membranes are subjected to immersion treatment in, for example, glycerin that is compatible with the hydrophilic liquid used for sealing and also has a high affinity for the hydrophobic separation membranes. Alternatively, the hydrophobic separation membranes are immersed in, for example, glycerin, and then the glycerin adhering to the hydrophobic separation membranes is replaced with an alcohol. Water can be selected as the liquid to be sealed for the hydrophobic separation membranes also in this case.

The temperature of the sealed liquid is not particularly specified. This is because sterilization can be performed when the temperature thereof can be increased to a prescribed temperature during steam sterilization. The smaller the difference between the temperature of the sealed liquid and the steam sterilization temperature is, the shorter the time required to increase the temperature to the steam sterilization temperature is. However, since the temperature of the sealed liquid sealed on the secondary side of the separation membranes is rapidly increased by the water vapor supplied, the difference in the time required to increase the temperature is small.

No particular limitation is imposed on the method of sealing the liquid on the secondary side of the separation membranes. An example when the liquid is water will be described below.

Water used as the liquid to be sealed is introduced into the primary side of the separation membrane module 2 to fill the primary side of the separation membrane module 2 with the water. Then pressure is applied to the primary side of the separation membrane module 2 or the water is sucked from the secondary side, whereby the water is filtered from the primary side to the secondary side, and the secondary side is filled with the water. Then the discharge valve 27 and the filtration valve 13 are closed to seal the water on the secondary side.

When the water is supplied from the primary side of the separation membrane module 2 to the secondary side by filtration, a sufficient amount of water may not be sealed on the secondary side of the separation membranes if the filtration time is short. Therefore, it is preferable to perform filtration for a prescribed time or longer.

For example, in a hollow fiber membrane module having an effective length of 1 m, after the primary side of the hollow fiber membranes is filled with water, filtration is performed for 15 minutes or longer at a filtration flux of 0.2 m/d. In this manner, 90% or more of the secondary side volume of the filtration portions of the hollow fiber membranes can be filled with the water.

It is preferable to use a high filtration flux when the liquid is sealed on the secondary side, because the liquid can be sealed at a fast rate and the operating time can be reduced and because air etc. in the pores of the separation membranes can be easily pushed out. For example, the filtration flux when the liquid is sealed on the secondary side is preferably 0.1 m/d or more and more preferably 0.2 m/d or more.

When water is sealed on the secondary side of the separation membrane module 2, it is preferable that the water be sealed in the largest possible volume on the secondary side of the filtration portions of the separation membranes. However, since filtration proceeds from portions of the separation membranes in which filtration resistance is small, gas such as air may remain on the secondary side. When the volume filled with water is increased, a problem arises in that the time required to introduce the water into the secondary side or the amount of introduced water increases. It is preferable for the separation membranes as a whole that a large volume be filled with water because the time required to increase temperature becomes short and drying of the membranes can be suppressed. The amount of water sealed on the secondary side of the separation membrane module 2 with respect to the secondary side volume of the filtration portions of the separation membranes is preferably 70% or more. If the amount is less than 70%, it is feared that the membranes may be partially dried.

The secondary side volume is the volume of the effective membrane areas of the separation membranes on their secondary side. For example, when a hollow fiber membrane module is used as the separation membrane module 2, the hollow fiber membranes are secured inside the separation membrane module 2 using an adhesive referred to as a potting agent. Portions of the hollow fiber membranes in potting layers are surrounded by the potting agent and therefore do not contribute to filtration. Thus these portions are not included in the effective membrane areas. Therefore, the volume of these portions is not counted as the secondary side volume.

Specifically, the secondary side volume of, for example, external pressure type hollow fiber membranes can be computed from the inner diameter of the hollow fiber membranes and the length of the effective membrane areas of the hollow fiber membranes. The external pressure type hollow fiber membranes generally have a circular cross section. However, even when the cross sectional shape is a triangle or a quadrilateral, the secondary side volume can be computed by simple calculation. The secondary side volume may be determined by sealing water on the secondary side of the separation membranes, discharging the water, and then measuring the amount of the discharged water. In this case, the volume of the effective membrane areas can be computed by subtracting the volume of water discharged together.

The amount of water sealed on the secondary side of the separation membranes can be measured as follows. After the supply of water to the separation membrane module 2 is stopped and then valves are manipulated to seal the water, the water on the primary side of the separation membrane module 2 is discharged. Then the water on the secondary side is discharged, and the amount of the discharged water is measured.

For example, after the water is filtered from the primary side of the separation membranes to the secondary side, the valves disposed on the secondary side are closed to seal the water on the secondary side. Then, after the water on the primary side of the separation membranes is discharged, the valves disposed on the secondary side are opened to discharge the water sealed on the secondary side while, for example, the secondary side is pressurized with air as needed. Then the amount of the discharged water is measured. In this case, the amount of water filling the liquid feed lines etc. on the secondary side is included in the measured amount. However, the amount of water on the secondary side of the hollow fiber membranes can be determined if the amount of water in the liquid feed lines is measured in advance.

Alternatively, the ratio of sealed water can be determined by observing the separation membranes from the primary side to measure the length of a portion in which water is sealed and the length of a portion in which air remains. It is desirable that the entire separation membranes can be observed. However, there are portions that cannot be observed visually. In this case, the separation membranes are observed partially, and the observed portions may be used as representative portions.

Alternatively, the amount of water sealed on the secondary side can be determined as follows. The mass of the separation membrane module 2 before water is sealed on the secondary side is measured in advance. Water is sealed on the secondary side, and water on the primary side is discharged. Then the mass of the separation membrane module 2 is measured. Also in this case, the amount of water filling the liquid feed lines etc. on the secondary side is included in the measured amount. However, the amount of water on the secondary side of the hollow fiber membranes can be determined if the amount of water in the liquid feed lines is measured in advance.

Water can be supplied to the secondary side by applying pressure to the primary side or subjecting the secondary side to suction before the primary side of the separation membranes is fully filled with the water. Since water can be sealed on the secondary side at a faster rate by filtering the water through the entire portion of the separation membranes, it is preferable to filter the water from the primary side to the secondary side after the entire primary side is filled with the water.

As described above, it is preferable to perform steam sterilization while the water sealed on the secondary side of the separation membrane module 2 is maintained in the sealed state.

When water vapor is introduced into the primary side of the separation membrane module 2 to perform steam sterilization, if the water vapor is introduced with the primary side filled with water, local heat exchange between the liquid water and the water vapor may occur abruptly. This causes vaporization of the water present on the primary side or condensation of the water vapor, so that the separation membranes vibrate. In this case, it is feared that the separation membranes and components of the separation membrane module 2 may be broken. Therefore, when sterilization is performed by introducing water vapor to the primary side of the separation membrane module 2, it is preferable that the amount of water on the primary side of the separation membrane module 2 be small.

However, when no liquid water is present on the primary side of the separation membrane module 2 and the pressure on the primary side is lower than the pressure on the secondary side, the water sealed on the secondary side may flow backward to the primary side. Therefore, although no particular limitation is imposed on the sealing method, it is necessary that, for example, the discharge valve 27 etc. disposed on the liquid feed lines on the secondary side be closed and that the pressure on the primary side be prevented from being lower than the pressure on the secondary side so that the amount of water sealed on the secondary side is maintained at 70% or more.

In the first embodiment, the vapor supply unit 20 supplies water vapor to the primary side of the separation membrane module 2. The temperature of the water vapor supplied to the separation membrane module 2 may be set to the sterilization temperature determined according to the characteristics of an object to be sterilized. Particularly, the temperature of the water vapor is preferably equal to or higher than 121° C. that is the same as the sterilization temperature of general steam sterilization. Preferably, ion exchanged water, water filtered through a reverse osmosis membrane, distilled water, or water having cleanliness equivalent to that of these types of water is used for the water vapor supplied. Water for the water vapor may be prepared by sterilizing ion exchanged water, water filtered through a reverse osmosis membrane, distilled water, etc. in advance and then vaporizing the resultant water to form the prescribed water vapor or by vaporizing ion exchanged water, water filtered through a reverse osmosis membrane, distilled water, etc. to form water vapor at a prescribed temperature and then subjecting the water vapor to sterilization treatment through, for example, a sterilization filter.

Sterilization of the separation membrane module 2 is performed by heating the separation membrane module to a prescribed temperature and then maintaining the temperature for a predetermined time. It is generally preferable that the sterilization be performed by heating the separation membrane module to 121° C. or higher and maintaining the temperature for 15 minutes to 20 minutes. Specifically, it is particularly preferable to perform sterilization by continuously supplying water vapor at 121° C. or higher to the separation membrane module 2 for 15 to 20 minutes. More specifically, the sterilization step may contain a heating step of increasing temperature and a temperature maintaining step of maintaining the temperature.

Whether or not the temperature of the separation membrane module is increased to an appropriate temperature during sterilization can be determined as follows.

For example, the correlation between the temperature of a fermenter 1 and the temperature of the separation membrane module 2 during steam sterilization is checked in advance. In this manner, by checking the temperature of the fermenter during sterilization, the temperature of the separation membrane module can be estimated indirectly.

The temperature of the separation membrane module 2 can also be checked by inserting a thermocouple into the separation membranes of the separation membrane module to measure the temperature during sterilization.

Alternatively, the correlation between the temperature of the surface of the casing of the separation membrane module 2 and the temperature inside the separation membrane module 2 is checked in advance. The temperature inside the separation membrane module 2 during sterilization can be estimated by measuring the surface temperature of the casing of the separation membrane module using, for example, a surface thermometer. In this manner, whether or not the internal temperature has reached a prescribed steam sterilization temperature can be checked.

Whether or not the conditions for steam sterilization such as temperature and time are appropriate can be determined by examining whether or not sterilization can be performed under these conditions in advance. This prior check can be performed as follows. A certain microorganism is placed on a portion of the separation membrane module 2 in which its temperature is less likely to increase (for example, a narrow portion between separation membranes), and then steam sterilization is performed. Then, for example, a culture medium containing a source of nutrient is supplied to the separation membrane module, and whether or not the microorganism grows is examined, whereby whether or not sterilization is performed appropriately can be checked.

When steam sterilization is performed, the separation membrane module 2 may be pre-heated in order to reduce a thermal load of the components of the separation membrane module.

For example, the separation membrane module 2 can be pre-heated by supplying warm water to the separation membrane module 2 through, for example, a liquid supply line 31 for supplying the liquid to be sealed. The warm water may be supplied, for example, through the pipe 23 for supplying the stock solution. The temperature of the warm water supplied to the separation membrane module 2 is preferably 40° C. to lower than 100° C. The pre-heating by supplying the warm water can reduce the time required to increase the temperature of the separation membrane module 2 when it is heated by supplying water vapor to 121° C. or higher, which is the sterilization temperature of general steam sterilization. The temperature of the warm water supplied is more preferably 80° C. to less than 100° C. The temperature of the warm water supplied may be gradually increased. For example, warm water at 20° C. is supplied at the beginning, and the temperature of the warm water may be increased gradually to about 80° C.

A separation membrane module 2 having a complicated shape has a portion into which the water vapor does not easily diffuse. However, when the separation membrane module 2 having such a shape is pre-heated with warm water, the time required to increase the temperature of the separation membrane module 2 after water vapor is supplied after pre-heating can be reduced.

When the separation membrane module 2 includes a component with low durability against steep temperature change, it is preferable that the temperature of the warm water supplied be gradually increased.

The warm water used is water prepared by heating water filtered through a reverse osmosis membrane, distilled water, or ion exchanged water using, for example, a heater. Since the warm water is used for sterilization, it is preferable that the warm water be used, for example, after filter sterilization through a filter. The filter used can be a commercial sterilization filter, and the trap diameter of the filter is preferably about 0.2 μm.

Water used as the warm water may be stored in, for example, a tank and fed to the separation membrane module 2. In this case, the water in the tank may be heated to a prescribed temperature in advance. Alternatively, a heat exchanger may be provided along the line to heat the water when the water is fed to the separation membrane module 2. The heat exchanger used may be a general heat exchanger such as a plat-type, tube-type, spiral-type, or double-pipe-type heat exchanger.

In the sterilization step (the heating step and the temperature maintaining step) and a cooling step after sterilization, water may be supplied to the secondary side of the separation membrane module 2, and the supplied water may be passed from the secondary side to the primary side. In the sterilization step and the cooling step after sterilization, when water vapor is supplied to the primary side while water is passed from the secondary side to the primary side, drying of the separation membranes can be suppressed.

In the cooling step after sterilization, warm water may be supplied from the secondary side to the primary side. By supplying warm water from the secondary side to the primary side, the potting layers heated to high temperature can be gradually cooled. In this manner, heat shock by abrupt cooling can be suppressed, and deterioration of the potting layers can be suppressed.

The water supplied to the secondary side of the separation membrane module 2 passes to the primary side of the separation membrane module 2 and is discharged from the separation membrane module 2 through the discharge line 33 and the discharge valve 32.

The water retained on the secondary side may be discharged directly in some types of separation membrane modules. In this case, the water is discharged through the discharge line 26 and the discharge valve 27 directly connected to the secondary side.

In the sterilization step, when water vapor is supplied to the primary side while water is passed from the secondary side to the primary side, it is preferable that the temperature and flow rate of the water supplied from the secondary side to the primary side be controlled such that the prescribed sterilization temperature is maintained during the sterilization step for the separation membrane module 2. If the temperature of the water supplied is low and its flow rate is high, the water passing from the secondary side of the separation membrane module 2 to the primary side may cause the temperature in the vicinity of the separation membranes of the separation membrane module 2 to become lower than the prescribed sterilization temperature. Therefore, it is preferable that the relation among the temperature and amount of supplied water, the temperature and amount of supplied water vapor, and the temperature of the separation membrane module 2 be examined in advance for the separation membrane module 2 used. Particularly, the flux of the water supplied to the separation membrane module 2 is preferably 0.001 to 1 m/d and more preferably 0.01 to 0.1 m/d. For example, when water vapor at 125° C. is supplied under the condition of a temperature of 121° C. or higher, it is not feared that such a flux will adversely affect the maintenance of the steam sterilization temperature because the water is heated to the prescribed steam sterilization temperature when the water supplied to the separation membranes passes therethrough.

The water may be supplied intermittently or continuously. However, in consideration of prevention of drying of the separation membranes and the stability of the temperature during sterilization, it is preferable to supply the water continuously.

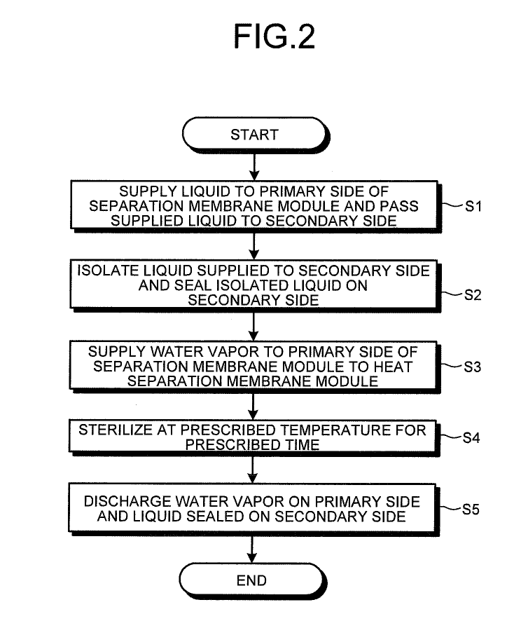

Referring next to FIG. 2, a method of sterilizing the separation membrane module 2 according to the first embodiment will be described. FIG. 2 is a flowchart for explaining sterilization treatment for the separation membrane module 2 according to the first embodiment.

In the sterilization treatment in the first embodiment, first, a liquid is supplied by the liquid supply unit 40 to the primary side of the separation membrane module 2 and passed to the secondary side (step S1). The liquid is supplied to the primary side of the separation membrane module 2 through the liquid supply line 31 by a liquid supply pump 21 with the discharge valve 27, the supply valve 19, a drainage valve 32, the filtration valve 13, and the circulation valve 17 being closed and with a liquid supply valve 22 being opened. The primary side of the separation membrane module 2 is filled with the liquid, and the liquid is then passed from the primary side to the secondary side. Preferably, the liquid is passed as follows. The filtration valve 13 is opened, and then the liquid is sucked by the filtration pump 11 from the secondary side until the secondary side is filled with the liquid to be sealed. The conditions under which the liquid to be sealed can be sealed in at least 70% of the volume of a space surrounded by portions of the separation membranes on the secondary side that are used for filtration, i.e., the secondary side volume of the filtration portions, are examined in advance. Examples of these conditions may include the amount of the liquid supplied by the liquid supply unit 40 and filtration flux. The temperature of the liquid supplied by the liquid supply unit 40 may be room temperature or a temperature higher than room temperature.

The liquid is supplied in an amount of at least 70% with respect to the secondary side volume of the filtration portions of the separation membrane module 2, and the secondary side is then isolated to seal the liquid on the secondary side (step S2). The liquid is sealed on the secondary side by closing the filtration valve 13. The filtration valve 13 is closed, and then the sealed liquid supply pump 21 is stopped to stop the supply of the liquid to the separation membrane module 2.

The liquid is sealed on the secondary side of the separation membrane module 2 (with the filtration valve 13 closed), and water vapor is then supplied to the primary side of the separation membrane module 2 by the vapor supply unit 20 to increase the temperature of the separation membrane module 2 to the prescribed sterilization temperature (step S3). When the water vapor is supplied, the circulation valve 17 and the liquid supply valve 22 are closed, and the supply valve 19 and the discharge valve 32 are opened, so that the water vapor is supplied to the primary side of the separation membrane module 2 through the pipe 34. The supply of the water vapor by the vapor supply unit 20 is continued while the water vapor is discharged through the discharge line 33 until the separation membrane module 2 is heated to the prescribed sterilization temperature. The liquid filling the primary side is discharged through the discharge line 33.

When water vapor is introduced into a portion in which a large amount of liquid water is present, an abrupt temperature change occurs due to contact between the water vapor and the liquid water, and this causes hammering to occur. Therefore, the water on the primary side may be discharged before the water vapor is introduced.

The pressure in the sterilization space must be maintained at the saturated water vapor pressure or higher so that prescribed temperature is achieved during steam sterilization. Therefore, a steam trap, for example, may be provided in the discharge line 33 so that only water (drain) formed by condensation of the water vapor can be discharged while the set pressure is maintained.

The separation membrane module 2 and another apparatus may be subjected to steam sterilization simultaneously, or the separation membrane module 2 alone may be subjected to steam sterilization with the pipe 25 for the crossflow filtration closed.

The water vapor is supplied by the vapor supply unit 20 and the separation membrane module 2 is heated to the prescribed sterilization temperature, and then the separation membrane module 2 is sterilized at the prescribed sterilization temperature for a prescribed time (step S4). In the sterilization using water vapor, the sterilization temperature is generally 121° C., and the sterilization time is generally 15 minutes to 20 minutes. However, the sterilization temperature and sterilization time may be appropriately changed according to the sterilization level required for the separation membrane module 2. To facilitate the maintenance of the temperature of the separation membrane module 2, water vapor is supplied in an amount that compensates for loss due to heat dissipation from the components of the separation membrane module 2. It is also preferable to thermally insulate the components of the sterilizing apparatus 100 to thereby reduce the amount of water vapor supplied.

A combination of the temperature increase in step S3 and the temperature maintenance in step S4 can be considered as the sterilization step.

After the sterilization treatment, the water vapor on the primary side of the separation membrane module 2 and the liquid sealed on the secondary side are discharged, and the sterilization treatment is completed (step S5). The water vapor on the primary side and the liquid sealed on the secondary side may be discharged through the discharge lines 26 and 33. The separation membrane module 2 may be left to cool to reduce the pressure of the water vapor on the primary side. Alternatively, the separation membrane module 2 may be cooled by supplying compressed air or cooling water. The liquid, particularly water, sealed on the secondary side may remain sealed to prevent drying of the separation membranes.

After the steam sterilization, it is feared that unsterilized substances in the outside air etc. may be mixed (sucked) when the inside of the steam sterilization object is in a negative pressure state, so it is preferable to avoid the negative pressure state as much as possible. Therefore, it is preferable that sterilized water or sterilized air be supplied after the steam sterilization to create positive pressure in the steam sterilization object.

In the first embodiment, after the high-boiling point liquid is sealed on the secondary side of the separation membrane module, water vapor is supplied to the primary side. In this manner, the liquid sealed on the secondary side transmits heat to the components of the separation membrane module 2 through the separation membranes, so that the time required to heat the separation membrane module 2 to the prescribed sterilization temperature can be significantly reduced. Particularly, when separation membranes with large pores are sterilized, since the sterilization is performed with the liquid sealed on the secondary side, the water vapor may pass from the primary side of the separation membranes to the secondary side. When the water vapor passes from the primary side to the secondary side, the water vapor spreads also over the secondary side, so that the separation membrane module 2 can be heated by the water vapor also from the secondary side. The time required to heat the separation membrane module 2 to the prescribed sterilization temperature can thereby be significantly reduced. Therefore, thermal deterioration of the potting agent etc. can be suppressed, and the frequency of replacement of the separation membrane module 2 can be reduced.

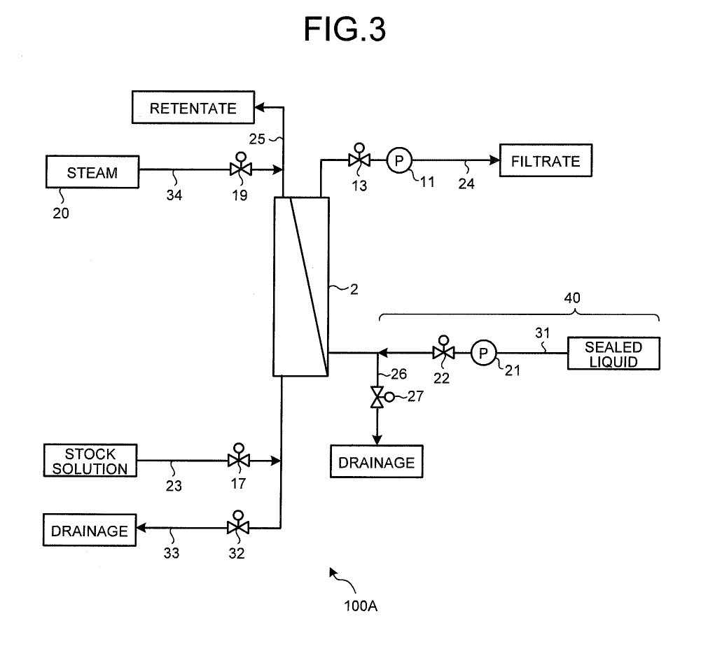

The liquid may be sealed directly on the secondary side of the separation membranes. FIG. 3 is a schematic diagram of a separation membrane module sterilizing apparatus according to a first modification of the first embodiment of the present invention. In a sterilizing apparatus 100A according to the first modification, the liquid supply unit 40 is connected to the secondary side of the separation membrane module 2. In the sterilizing apparatus 100A, the liquid supply unit 40 is connected to the discharge line 26, and the liquid is supplied directly from the liquid supply unit 40 to the secondary side of the separation membrane module 2 with the discharge valve 27 closed. When air is present on the secondary side, it is feared that air lock may prevent the liquid from being sealed. Therefore, the filtration valve 13 is opened.

After the filling ratio of the secondary side with the liquid supplied thereto by the liquid supply unit 40, i.e., the liquid filling ratio of the space surrounded by portions on the secondary side that are used for filtration, reaches 70% or more, the filtration valve 13 and the liquid supply valve 22 are closed, whereby the liquid is sealed on the secondary side. Alternatively, the secondary side is first filled with the liquid, and then the liquid may be subjected to counter pressure filtration from the secondary side of the separation membranes to the primary side. By closing the liquid supply valve 22 etc. on the secondary side of the separation membrane module 2 after the liquid was filtered from the secondary side of the separation membrane to the primary side, the liquid is sealed on the secondary side.

In the sterilization step after the counter pressure filtration, the liquid outflow paths on the secondary side, for example, the filtration valve 13, the discharge valve 27, and the liquid supply valve 22 in FIG. 3, are closed so that the liquid supplied to the secondary side does not flow out. The sterilization step may be performed with the filtration valve 13 and the discharge valve 27 being closed and the liquid supply valve 22 being opened, i.e., while the liquid is supplied to the secondary side.

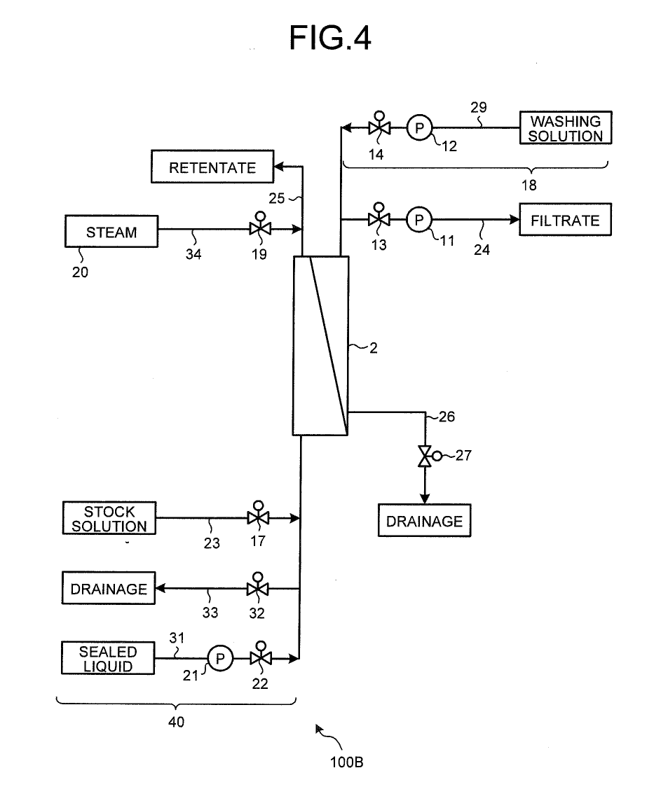

When a solvent other than water is used as the liquid to be sealed, it is preferable to use a sterilizing apparatus shown in FIG. 4. FIG. 4 is a schematic diagram of the separation membrane module sterilizing apparatus according to a second modification of the first embodiment of the present invention. A sterilizing apparatus 100B includes a separation membrane washing unit 18 for supplying a washing solution to the secondary side of the separation membrane module 2.

The separation membrane washing unit 18 includes a washing solution tank, a washing solution supply pump 12, and a washing solution valve 14. The separation membrane washing unit 18 supplies the washing solution from the washing solution tank to the secondary side of the separation membrane module 2 through a washing solution supply line 29 when the washing solution supply pump 12 is actuated. In the second modification of the first embodiment, the liquid sealed on the secondary side is discharged in the sterilization treatment according to the first embodiment (step S5), and then the washing solution is supplied to the secondary side of the separation membrane module 2 by the separation membrane washing unit 18. When the washing solution is supplied, the discharge valve 27 and the filtration valve 13 are closed, and the washing solution valve 14 is opened. After the secondary side was filled with the washing solution, a valve on the primary side such as the discharge valve 32 is opened, whereby the washing solution is also passed from the secondary side to the primary side. The supplied washing solution can wash the liquid remaining on the primary and secondary sides of the separation membrane module 2. The separation membrane washing unit 18 may be connected to the primary side to supply the washing solution from the primary side to the secondary side. For example, the washing solution is supplied to the primary side of the separation membrane module 2 and filtered to the secondary side of the separation membranes to perform washing.

Water can be preferably used as the washing solution. The washing solution may be water to which an alkali, an acid, an oxidizing agent, or a reducing agent used for backwashing of the separation membrane module 2 is added.

In the second modification, when a solvent other than water is used as the liquid to be sealed, the discharge line 26 may be connected to the liquid supply unit 40 so that the sealed liquid is re-used. The sterilization step may be performed with the filtration valve 13 and the discharge valve 27 being closed and the washing solution valve 14 being opened, i.e., while the washing solution is supplied to the secondary side.

### Second Embodiment

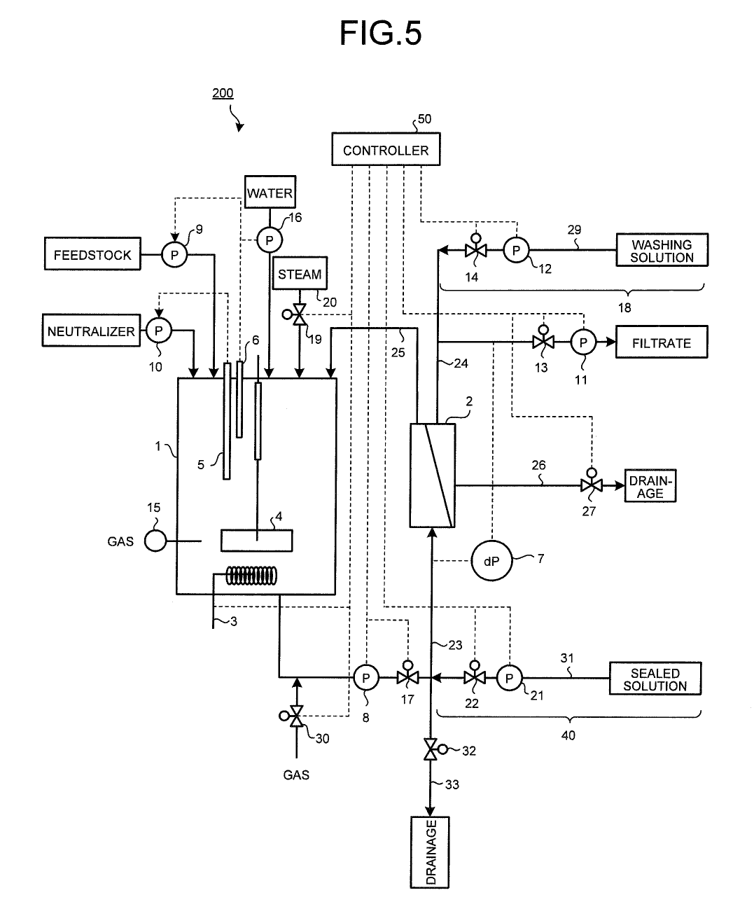

Referring next to FIG. 5, a second embodiment of the present invention will be described. FIG. 5 is a schematic diagram of a membrane separation-type continuous fermentation apparatus according to the second embodiment of the present invention.

A membrane separation-type continuous fermentation apparatus 200 includes: a fermenter 1 for converting a fermentation feedstock to a fermented liquid containing a chemical by fermentation cultivation using a microorganism; a separation membrane module 2 for separating the chemical from the fermented liquid; a circulation pump 8 for supplying the fermented liquid to the separation membrane module 2; a vapor supply unit 20 for supplying water vapor for steam sterilization; a liquid supply unit 40 for supplying a liquid to be sealed, to the secondary side of the separation membrane module 2; and a controller 50 for controlling the respective components.

The feedstock and a microorganism or cultured cells are fed to the fermenter 1 by a feedstock supply pump 9. A fermentation step proceeds in the fermenter 1. The membrane separation-type continuous fermentation apparatus 200 includes a stirrer 4 and a gas supply unit 15. The stirrer 4 stirs the fermented liquid in the fermenter 1. The gas supply unit 15 can supply a required gas. In this case, the supplied gas may be collected, recycled, and again supplied by the gas supply unit 15.

The membrane separation-type continuous fermentation apparatus 200 includes a pH sensor-controller 5 and a neutralizer supply pump 10. The pH sensor-controller 5 detects the pH of a culture solution and then controls the neutralizer supply pump 10 according to the detection results such that the culture solution exhibits a pH within a set range. The neutralizer supply pump 10 is connected to a tank for an acidic aqueous solution and a tank for an alkaline aqueous solution, and the pH of the culture solution is controlled by adding one of the aqueous solutions to the fermenter 1. Since the pH of the culture solution is maintained within a certain range, fermentation production can be performed with high productivity. The neutralizers, i.e., the acidic aqueous solution and the alkaline aqueous solution, correspond to pH adjusters.

The circulation pump 8 feeds the culture solution, i.e., the fermented liquid, in the apparatus from the fermenter 1 to the separation membrane module 2 to circulate the flow of the unfiltered fermented liquid from the separation membrane module 2 to the fermenter 1 by the crossflow filtration. The circulation pump 8 feeds the fermented liquid to the separation membrane module 2 through the circulation valve 17 and the pipe 23 to circulate the unfiltered fermented liquid not filtered through the separation membrane module 2 to the fermenter 1 through the pipe 25. The fermented liquid containing a chemical, i.e., a fermentation product, is filtered through the separation membrane module 2 and separated into the microorganism and the chemical, i.e., the fermentation product, and the chemical is taken out of the apparatus system as the filtrate. The separated microorganism remains present in the apparatus system, and therefore the concentration of the microorganism in the apparatus system is maintained at a high level. This allows fermentation production with high productivity.

The separation membrane module 2 is connected to the fermenter 1 through the circulation pump 8. Preferably, filtration by the separation membrane module 2 is performed under suction by the filtration pump 11. The filtrate filtered through the separation membrane module 2 is discharged and collected from the filtrate discharge line 24 through the filtration valve 13. The membrane separation-type continuous fermentation apparatus 200 may include a differential pressure sensor-controller 7 for detecting pressure difference in the separation membranes of the separation membrane module 2. Stable filtration can be performed by controlling the filtration pump 11 while the pressure difference in the separation membranes of the separation membrane module 2 is detected by the differential pressure sensor-controller 7. The filtration pump 11 is controlled such that the value of the pressure difference in the separation membranes of the separation membrane module 2 is within a certain range. Filtration may be performed only through the pressure by the circulation pump 8 without suction by the filtration pump 11 and without using special power. The amount of the fermented liquid fed from the fermenter 1 to the separation membrane module 2 can be appropriately controlled by controlling the output of the circulation pump 8.

The fermenter 1 may include a temperature controller 3. The temperature controller 3 includes a temperature sensor for detecting temperature, a heating unit and/or a cooling unit, and a control unit. The temperature controller 3 uses the temperature sensor to detect the temperature inside the fermenter 1 and controls the heating unit and/or the cooling unit through the control unit according to the detection results such that the temperature is within a certain range to thereby control the temperature inside the fermenter 1. In this manner, the temperature of the fermenter 1 is maintained constant, and the concentration of the microorganism is thereby maintained at a high level.

The correlation between the temperature of the fermenter 1 and the temperature of the separation membrane module 2 during steam sterilization is examined in advance. This allows the temperature of the separation membrane module to be indirectly estimated by checking the temperature of the fermenter during steam sterilization.

Water may be added directly or indirectly to the fermenter 1. A water supply unit supplies water directly to the fermenter 1 and is specifically composed of a water supply pump 16. Indirect water supply includes supply of the feedstock, addition of a pH adjuster, etc. Preferably, materials added to the membrane separation-type continuous fermentation apparatus 200 have been sterilized in order to prevent fouling by contaminants and to perform fermentation efficiently. For example, a culture medium may be sterilized by heating after the raw materials of the culture medium are mixed. If necessary, the culture medium, the pH adjusters, and water added to the fermenter may be sterilized, for example, by passing them through a sterilization filter.

A level sensor-controller 6 includes a sensor for detecting the level of the liquid in the fermenter 1 and a control unit. The control unit controls the feedstock supply pump 9, the water supply pump 16, etc. according to the detection results from the sensor. The amounts of liquids flowing into the fermenter 1 are thereby controlled, so that the liquid level in the fermenter 1 is maintained within a certain range.

The separation membrane washing unit 18 includes a washing solution tank, a washing solution supply pump 12, and a washing solution valve 14. The separation membrane washing unit 18 supplies a washing solution from the washing solution tank to the secondary side of the separation membrane module 2 when the washing solution supply pump 12 is activated, whereby backwashing is performed. The backwashing is a method of removing foulants accumulated on the surfaces of the separation membranes by feeding the washing solution from a filtrate side, i.e., the secondary side, of the separation membranes, to a fermented liquid side, i.e., the primary side. The washing solution supplied to the secondary side of the separation membrane module 2 passes through the separation membranes to be filtered to the primary side. By supplying the washing solution to the separation membrane module 2, the separation membranes are washed. When the backwashing is performed, the washing solution is supplied to the separation membrane module 2 with the filtration valve 13 disposed between the separation membrane module 2 and the filtration pump 11 being closed and filtration in the separation membrane module 2 being stopped. During the backwashing, the circulation pump 8 may be operated or stopped. When the backwashing is performed while the circulation pump 8 is operated, the pressure by the washing solution supply pump 12 may be set to be higher than the sum of the pressure by the circulation pump 8 and the pressure difference in the separation membranes.

An alkali, an acid, an oxidizing agent, or a reducing agent may be added to the washing solution used for the backwashing so long as the fermentation is not significantly inhibited. Examples of the alkali may include sodium hydroxide and calcium hydroxide. Examples of the acid may include oxalic acid, citric acid, hydrochloric acid, and nitric acid. Examples of the oxidizing agent may include hypochlorites and hydrogen peroxide. Examples of the reducing agent may include inorganic reducing agents such as sodium hydrogen sulfite, sodium sulfite, and sodium thiosulfate.

The transmembrane pressure difference when the fermented liquid including a microorganism or cultured cells is filtered through the separation membranes in the separation membrane module 2 may be set such that the separation membranes are not easily clogged with the microorganism, the cultured cells, and the components of the culture medium. For example, the filtration treatment can be performed by setting the transmembrane pressure difference within the range of 0.1 kPa or more and 20 kPa or less. The transmembrane pressure difference is preferably within the range of 0.1 kPa or more and 10 kPa or less and more preferably within the range of 0.1 kPa or more and 5 kPa or less. When the transmembrane pressure difference is within the above range, clogging with a microorganism (particularly a prokaryote) and the components of the culture medium and a reduction in the amount of water permeation can be suppressed, so that the occurrence of a problem during operation of continuous fermentation can be effectively suppressed.

The vapor supply unit 20 supplies water vapor to the fermenter 1, the separation membrane module 2, and peripheral pipes through the supply valve 19. The water vapor is supplied to the components of the membrane separation-type continuous fermentation apparatus 200 through the supply valve 19 to perform sterilization of the apparatus under prescribed steam sterilization conditions. After the steam sterilization, compressed air may be supplied to the membrane separation-type continuous fermentation apparatus 200 through a gas supply valve 30. The water vapor is thereby discharged from the fermenter 1 etc., and the fermenter 1 is cooled.

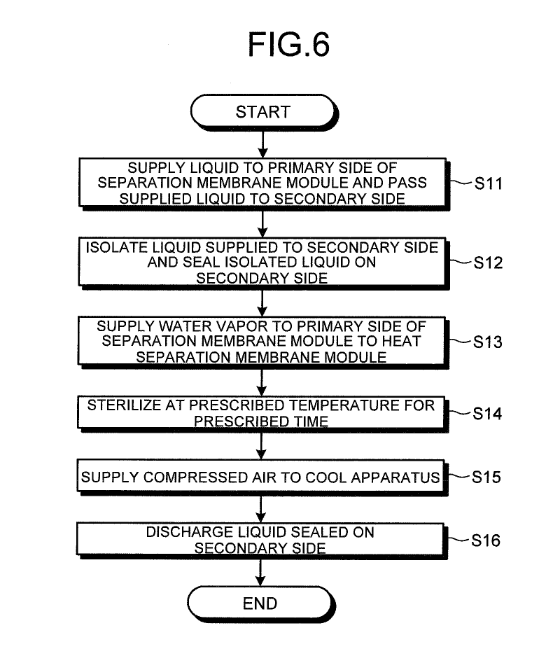

Referring next to FIG. 6, a method of sterilizing the separation membrane module 2 according to the second embodiment will be described. FIG. 6 is a flowchart for explaining sterilization treatment for the separation membrane module 2 according to the second embodiment.

In the method of sterilizing the separation membrane module 2 according to the second embodiment, as in the first embodiment, first, a liquid to be sealed on the secondary side is supplied by the liquid supply unit 40 to the primary side of the separation membrane module 2 and passed to the secondary side (step S11). Specifically, the primary side of the separation membrane module 2 is filled with the liquid through the liquid supply line 31 by the sealed liquid supply pump 21 with the discharge valve 27, the circulation valve 17, the filtration valve 13, and the washing solution valve 14 being closed and with the sealed liquid supply valve 22 being opened. Then filtration is performed by operating the filtration pump 11 with the filtration valve 13 being opened, and the liquid is thereby passed to the secondary side of the separation membrane module 2. The filtration to the secondary side is performed until at least 70% of the volume of a space surrounded by portions of the separation membranes on the secondary side that are used for the filtration, i.e., the secondary side volume of the filtration portions, is filled with the liquid to be sealed. Preferably, the time required for at least 70% of the secondary side volume to be filled with the liquid is examined in advance by a test.

The liquid is supplied in an amount of at least 70% with respect to the secondary side volume of the filtration portions of the separation membrane module 2, and then the secondary side is isolated to seal the liquid on the secondary side (step S12). The liquid supply pump 21, the washing solution supply pump 12, and the filtration pump 11 are stopped to stop the supply of the liquid to the separation membrane module 2. The filtration valve 13, the washing solution valve 14, the sealed liquid supply valve 22, and the discharge valve 27 are closed to seal the liquid on the secondary side of the separation membrane module 2.

After the liquid is sealed on the secondary side of the separation membrane module 2, water vapor is supplied by the vapor supply unit 20 to the primary side of the separation membrane module 2 and the components of the membrane separation-type continuous fermentation apparatus 200 such as the fermenter 1 to thereby heat the components of the membrane separation-type continuous fermentation apparatus 200 including the separation membrane module 2 to a prescribed sterilization temperature (step S13). The discharge valve 32 is opened to discharge water on the primary side of the separation membrane module 2 and in the pipe 23 etc. from the discharge line 33. Then the supply valve 19 and the circulation valve 17 are opened to supply water vapor from the vapor supply unit 20 to the fermenter 1, the separation membrane module 2, etc. to thereby increase the temperature of the separation membrane module 2.

Condensed water (drain) generated when the membrane separation-type continuous fermentation apparatus 200 is subjected to steam sterilization may be discharged, for example, from the discharge line 33 by opening the discharge valve 32. In this case, a steam trap, for example, may be provided in the discharge line 33 so that the pressure of the water vapor is maintained constant.

After the heating step, the components of the membrane separation-type continuous fermentation apparatus 200 including the separation membrane module 2 are sterilized at the prescribed sterilization temperature for a prescribed time (step S14). When the fermenter 1, the pipes 23 and 25, etc. are subjected to steam sterilization at the same time, the temperature of the supplied water vapor is set such that a place in which its temperature is most difficult to increase is heated to a temperature equal to or higher than the prescribed steam sterilization temperature. The supply time of water vapor is set such that the time after the place in which its temperature is most difficult to increase is heated to the prescribed steam sterilization temperature or higher is equal to or longer than the prescribed steam sterilization time. Generally, the temperature of the supplied water vapor is set to preferably 121° C. or higher with heat dissipation measures such as thermal insulation taken.

After completion of the sterilization treatment, the gas supply valve 30 is opened, and compressed air is supplied to the respective components of the membrane separation-type continuous fermentation apparatus 200 including the primary side of the separation membrane module 2 to cool the membrane separation-type continuous fermentation apparatus 200 (step S15). Natural cooling may be performed without supply of air. However, when a component with insufficient heat resistance is used, it is preferable to leave the membrane separation-type continuous fermentation apparatus 200 to cool while compressed air is supplied in order to prevent the service life from being shortened and to prevent partial cooling that causes a partial reduction in pressure. During cooling of the membrane separation-type continuous fermentation apparatus 200 using compressed air, the compressed air may be blown with the liquid sealed on the secondary side of the separation membrane module 2. When the compressed air is blown with no liquid sealed on the secondary side, the pores of the separation membranes are dried. In this case, it may be necessary to subject the pores of the separation membranes to replacement treatment with a liquid, in order to perform filtration treatment. In the second embodiment, cooling treatment by blowing compressed air is performed with the liquid sealed on the secondary side, and this can suppress drying of the pores of the separation membranes.

After the separation membrane module 2 is cooled, the liquid sealed on the secondary side is discharged as needed, and the sterilization treatment is completed (step S16).

In the second embodiment, as in the first embodiment, the high-boiling point liquid is sealed on the secondary side of the separation membrane module 2, and water vapor is then supplied to the primary side. In this case, the liquid sealed on the secondary side transmits heat from the water vapor to the respective components of the separation membrane module 2 through the separation membranes. Therefore, the time required for the separation membrane module 2 to be heated to the prescribed sterilization temperature can be significantly reduced, and thermal deterioration of the potting agent etc. can thereby be suppressed. Particularly, when separation membranes with large pores are sterilized, since the sterilization is performed with the liquid sealed on the secondary side, the water vapor may pass from the primary side of the separation membranes to the secondary side. When the water vapor passes from the primary side to the secondary side, the water vapor spreads also over the secondary side, so that the separation membrane module 2 can be heated by the water vapor also from the secondary side. The time required to heat the separation membrane module 2 to the prescribed sterilization temperature can thereby be significantly reduced.

In the second embodiment, after the sterilization treatment, compressed air is supplied with the liquid sealed on the secondary side so that negative pressure is not generated, and then the membrane separation-type continuous fermentation apparatus 200 is left to cool, so that drying of the pores of the separation membranes in the separation membrane module 2 can be suppressed. In this manner, filtration treatment can be performed immediately after the sterilization treatment without performing extra treatment such as replacement with the liquid phase for the separation membranes.

In the second embodiment, after the sterilization treatment, compressed air is supplied with the liquid sealed on the secondary side, and the membrane separation-type continuous fermentation apparatus 200 is left to cool. Therefore, hammering due to rapid condensation of water vapor and contamination with germs can also be suppressed.

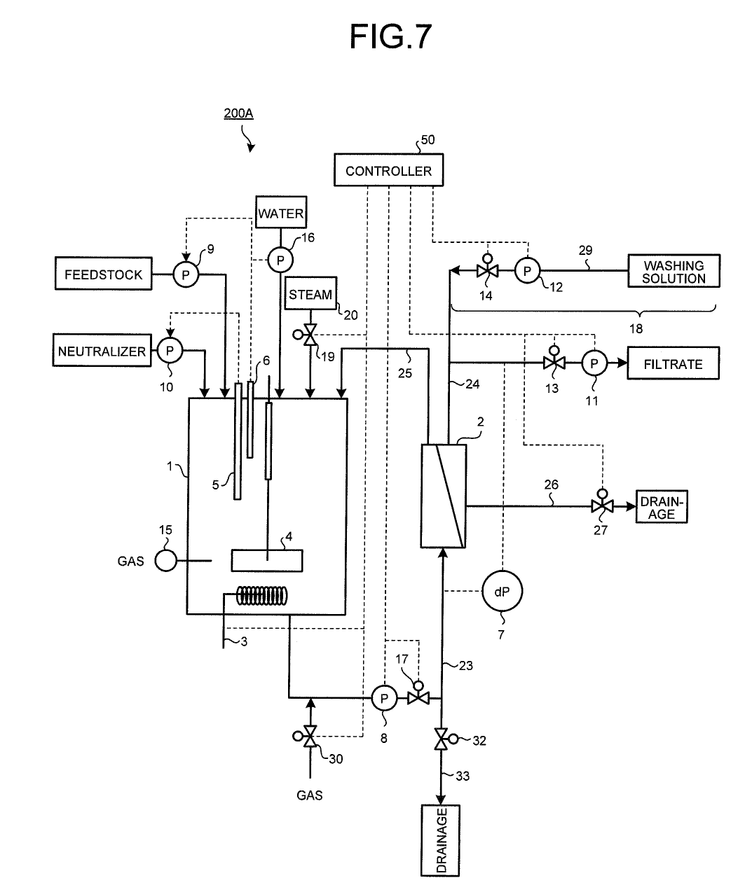

The membrane separation-type continuous fermentation apparatus 200 according to the second embodiment of the present invention is configured to include the liquid supply unit 40. However, when water is used as the liquid to be sealed, the liquid to be sealed (water) can be sealed on the secondary side of the separation membrane module 2 without providing the liquid supply unit 40. FIG. 7 is a schematic diagram of a membrane separation-type continuous fermentation apparatus 200A according to a modification of the second embodiment. When steam sterilization is performed on the membrane separation-type continuous fermentation apparatus 200A, water is sealed on the secondary side of the separation membrane module 2 as follows. First, the water supply pump 16 is actuated to supply water to the fermenter 1, and then the water in the fermenter 1 is circulated to the separation membrane module 2 using the circulation pump 8. While the water is circulated by the circulation pump 8, the filtration valve 13 is opened, and the filtration pump 11 is operated to perform filtration, so that the water is passed to the secondary side of the separation membrane module 2. The secondary side of the separation membrane module 2 is filled with the water, and then the filtration pump 11 is stopped, and the filtration valve 13 is closed, whereby the water can be sealed on the secondary side. After the water was sealed on the secondary side, water in the fermenter 1, the pipes 23 and 25, etc. and on the primary side of the separation membrane module 2 is discharged, and then steam sterilization is performed in the manner described in the second embodiment. In this manner, the time for heating to the sterilization temperature can be shortened.

In the membrane separation-type continuous fermentation apparatus 200 according to the second embodiment of the present invention, the liquid supply unit 40 may be connected to the secondary side of the separation membrane module 2. When the liquid supply unit 40 is connected to the secondary side of the separation membrane module 2, the liquid supply unit 40 may be connected to the filtrate discharge line 24 to which the separation membrane washing unit 18 is connected. When the liquid supply unit 40 is connected to the secondary side, the liquid is supplied by the liquid supply unit 40 to the secondary side to seal the liquid on the secondary side. In addition, when the membrane separation-type continuous fermentation apparatus is cooled by compressed air after completion of steam sterilization, the liquid may be continuously supplied from the liquid supply unit 40 to the separation membrane module 2 to perform cooling while the liquid is subjected to counter pressure filtration from the secondary side to the primary side.

The microorganism and cultured cells used in the membrane separation-type continuous fermentation apparatus 200 according to the present embodiment will be described. No particular limitation is imposed on the microorganism and cultured cells used in the present embodiment. Examples of the microorganism may include: yeasts often used in fermentation industry such as baker's yeast; eukaryotic cells such as filamentous fungi; and prokaryotic cells such as Escherichia coli, lactic acid bacteria, coryneform bacteria, and actinobacteria. Examples of the cultured cells may include animal cells and insect cells. The microorganism and cultured cell used may be those isolated from the natural environment or those partially modified in their nature by mutation or gene recombination.

When lactic acid is produced, it is preferable to use yeast when an eukaryotic cell is use or to use a lactic acid bacterium when a prokaryotic cell is used. Of these, the yeast is preferably yeast obtained by introducing a lactate dehydrogenase-coding gene into its cell. The lactic acid bacterium used is preferably a lactic acid bacterium capable of producing lactic acid at 50% or more as a yield per sugar with respect to consumed glucose and is more preferably a lactic acid bacterium capable of producing lactic acid at 80% or more of a yield per sugar.

The fermentation feedstock used in the present embodiment may be any fermentation feedstock that can facilitate the growth of the microorganism or cultured cells to be cultured and can allow a fermentation product, i.e., the target chemical, to be preferably produced. A liquid culture medium is used as the fermentation feedstock. A material which is a component of the culture medium and is converted to the target chemical (i.e., a feedstock in a narrow sense) may be referred to as a feedstock. However, in the present description, unless otherwise mentioned, the culture medium as a whole is referred to as a feedstock. The feedstock in a narrow sense is, for example, a saccharide such as glucose, fructose, or sucrose, each of which is a fermentation substrate used to obtain a chemical, i.e., an alcohol.

The feedstock appropriately contains a carbon source, a nitrogen source, inorganic salts and, if necessary, organic micronutrients such as amino acids and vitamins. The carbon source used is any of: saccharides such as glucose, sucrose, fructose, galactose, and lactose; solutions obtained by saccharification of starch and containing any of these saccharides; sweet potato molasses; beet molasses; high-test molasses; organic acids such as acetic acid; alcohols such as ethanol; and glycerin. The nitrogen source used is any of ammonia gas, ammonia water, ammonium salts, urea, nitrates, and other organic nitrogen sources used adjunctively such as oil cakes, soybean hydrolysate, casein decomposition products, other amino acids, vitamins, corn steep liquor, yeasts or yeast extracts, meat extracts, peptides such as peptone, various fermentation bacterial cells, and hydrolysates thereof. The inorganic salt added may be any of phosphates, magnesium salts, calcium salts, iron salts, and manganese salts.

When a specific nutrient is necessary for the growth of the microorganism or cultured cells, the nutrient is added to the feedstock as a preparation or a natural product containing the nutrient. The feedstock may contain an antifoaming agent as needed.

In the present description, the culture solution is a solution obtained as a result of proliferation of the microorganism or cultured cells in the fermentation feedstock. In continuous fermentation, the fermentation feedstock may be added to the culture solution. However, the composition of the fermentation feedstock added may be appropriately changed from the composition at the beginning of the cultivation so that the productivity of the target chemical increases. For example, the concentration of the fermentation feedstock in a narrow sense, the concentrations of other components in the culture medium, etc. can be changed.

In the present description, the fermented liquid is a liquid containing a material produced as a result of fermentation and may contain the feedstock, the microorganism or cultured cells, and the chemical. In other words, the terms “culture solution” and “fermented liquid” may be used with substantially the same meaning.

With the membrane separation-type continuous fermentation apparatus 200 according to the second embodiment, a chemical, i.e., a converted material, is produced in the fermented liquid by the microorganism or cultured cells described above. Examples of the chemical may include materials mass-produced in the fermentation industry, such as alcohols, organic acids, amino acids, and nucleic acids. Examples of the alcohols may include ethanol, 1,3-butanediol, 1,4-butanediol, and glycerol. Examples of the organic acids may include acetic acid, lactic acid, pyruvic acid, succinic acid, malic acid, itaconic acid, and citric acid. Examples of the nucleic acids may include inosine, guanosine, and cytidine. The method of the present invention can also be applied to production of materials such as enzymes, antibiotic substances, and recombinant proteins.

The membrane separation-type continuous fermentation apparatus 200 according to the second embodiment can be applied to production of a chemical product, a dairy product, a medical product, a food product, or a brewed product. Examples of the chemical product may include organic acids, amino acids, and nucleic acids. Examples of the dairy product may include low-fat milk. Examples of the food product may include lactic acid beverages. Examples of the brewed product may include beer and shochu, or Japanese distilled spirit. The enzymes, antibiotic substances, recombinant proteins, etc. produced by the method of the present invention are applicable to medical products.

In the production of a chemical by continuous fermentation, the continuous fermentation (i.e., extraction of the culture solution) may be started after batch cultivation or fed-batch cultivation is performed in the initial stage of cultivation to increase the concentration of the microorganism. Alternatively, after the concentration of the microorganism is increased, high-concentration bacterial cells may be seeded to start cultivation and perform continuous fermentation simultaneously. In the production of the chemical by continuous fermentation, supply of the feedstock culture solution and extraction of the culture solution can be started at an appropriate time. The supply of the feedstock culture solution and the extraction of the culture solution are not necessarily started at the same time. The supply of the feedstock culture solution and the extraction of the culture solution may be performed continuously or intermittently.

A nutrient necessary for proliferation of the bacterial cells may be added to the culture solution to allow continuous proliferation of the bacterial cells. In an embodiment preferred for obtaining efficient productivity, the concentration of the microorganism or cultured cells in the culture solution is maintained at a high level within such a range that the environment of the culture solution is not unsuitable for proliferation of the microorganism or cultured cells so that the death rate of the microorganism or cultured cells does not become high. The concentration of the microorganism or cultured cells in the culture solution during, for example, D-lactic acid fermentation using an SL-lactic acid bacterium is such that the concentration of the microorganism on a dry weight basis is maintained at 5 g/L or more, so that good production efficiency is obtained.

When a saccharide is used as the feedstock for the production of a chemical by continuous fermentation, it is preferable that the concentration of the saccharide in the culture solution be maintained at 5 g/L or less. The reason that it is preferable to maintain the concentration of the saccharide in the culture solution at 5 g/L or less is that the loss of the saccharide due to extraction of the culture solution is minimized.

The cultivation of the microorganism and cultured cells is generally performed within the pH range of 3 or more and 8 or less and the temperature range of 20° C. or higher and 60° C. or lower. Generally, the pH of the culture solution is adjusted in advance to a prescribed pH value of 3 or more and 8 or less with an inorganic or organic acid, an alkaline material, urea, calcium carbonate, ammonia gas, etc. When it is necessary to increase the supply rate of oxygen, means such as addition of oxygen to air to maintain the oxygen concentration at 21% or higher, pressurization of the culture solution, increasing the rate of stirring, or increasing the amount of airflow is used.

During the operation of continuous fermentation, it is preferable to monitor the concentration of the microorganism in the microorganism fermenter. The concentration of the microorganism can be measured by collecting a sample and measuring the concentration in the sample. However, preferably, a microorganism concentration sensor such as an MLSS sensor is provided in the microorganism fermenter to monitor the change in the microorganism concentration continuously.

In the production of a chemical by continuous fermentation, the culture solution and the microorganism or cultured cells can be extracted from the fermenter as needed. For example, when the concentration of the microorganism or cultured cells in the fermenter is excessively high, clogging of the separation membranes is more likely to occur. Therefore, extraction of the microorganism or cultured cells can prevent clogging from occurring. The performance of production of the chemical may vary according to the concentration of the microorganism or cultured cells in the fermenter. However, by extracting the microorganism or cultured cells on the basis of the production performance used as an indicator, the production performance can be maintained.

In the production of a chemical by continuous fermentation, the number of fermenters for continuous cultivation operation performed while fresh bacterial cells having a fermentation production ability are proliferated is not limited so long as a continuous cultivation method in which a product is produced while the bacterial cells are proliferated is performed. In the production of a chemical by continuous fermentation, it is generally preferable in terms of control of the cultivation that the continuous cultivation operation be performed in a single fermenter. A plurality of fermenters can be used when, for example, the volume of the fermenters is small. In this case, even when the continuous fermentation is performed using a plurality of fermenters connected in parallel or series through piping, the fermentation product can be obtained with high productivity.

### EXAMPLES

The effects of the present invention will be described in more detail by way of Examples, but the present invention is not limited to the following Examples.

### Reference Example 1

### Production of Hollow Fiber Membranes

A vinylidene fluoride homopolymer having a weight average molecular weight of 417,000 and γ-butyrolactone were dissolved in respective ratios of 38% by mass and 62% by mass at a temperature of 170° C. The obtained macromolecular solution was discharged from a nozzle while γ-butyrolactone serving as a hollow section-forming liquid accompanied the macromolecular solution and was then solidified in a cooling bath containing an aqueous solution of 80% by mass of γ-butyrolactone at a temperature of 20° C. to produce hollow fiber membranes having a spherical structure. Then 14% by mass of a vinylidene fluoride homopolymer having a mass average molecular weight of 284,000, 1% by mass of cellulose acetate propionate (CAP482-0.5, manufactured by Eastman Chemical Company), 77% by weight of N-methyl-2-pyrrolidone, 5% by mass of polyoxyethylene coconut oil fatty acid sorbitan (product name: IONET (registered trademark) T-20C, manufactured by Sanyo Chemical Industries, Ltd.), and 3% by mass of water were mixed and dissolved at a temperature of 95° C. to prepare a macromolecular solution. This membrane-forming stock solution was uniformly applied to the surface of the hollow fiber membranes having a spherical structure and immediately solidified in a water bath to produce hollow fiber membranes in which a three-dimensional network structure was formed on a spherical structure layer. The average pore diameter of the obtained hollow fiber membranes on their surface on a to-be-treated water side was 0.05 μm.

### Reference Example 2

### Production of Separation Membrane Module 2

A separation membrane module 2 was produced using a molded product, i.e., a polysulfone resin-made tubular container (inner diameter: 35 mm), as the case of the separation membrane module. The hollow fiber membranes produced in Reference Example 1 were used as the separation membranes and brought into contact with saturated water vapor at 121° C. for 1 hour. To bring the hollow fiber membranes into contact with saturated water vapor, an autoclave “LSX-700” manufactured by TOMY SEIKO Co., Ltd. was used. 325 hollow fiber membranes (outer diameter: 1.4 mm, effective length: 20 cm) were inserted into the above-described module case, and the module case and the hollow fiber membranes were bonded at their opposite ends using urethane resins (SA-7068A/SA-7068B, manufactured by SANYU REC Co., Ltd., the two resins were mixed at a weight ratio of 64:100). At each of the opposite ends of the hollow fiber membranes of the separation membrane module 2, an excess bonded portion was cut so as to open the hollow fiber membranes. The filling ratio of the hollow fiber membranes in the separation membrane module 2 was 50%. The separation membrane module 2 has a structure including a nozzle provided in a lateral lower portion of the separation membrane module 2, a nozzle provided in a lateral upper portion of the separation membrane module 2, and nozzles provided, respectively, at the upper and lower ends of the separation membrane module 2, and a fluid flows into/is discharged from the interior of the hollow fiber membranes through the upper or lower end of the separation membrane module. The fluid flows into/is discharged from the exterior of the hollow fiber membranes through the nozzle provided in the lateral lower or upper portion of the separation membrane module. An 80% aqueous ethanol solution was supplied to the primary side of the module case. Then part of the 80% aqueous ethanol solution was filtered from the secondary side to fill the separation membrane module 2 with the 80% aqueous ethanol solution, and the resultant separation membrane module 2 was left to stand for 1 hour. Then the 80% aqueous ethanol solution was discharged, and the separation membrane module 2 was washed and replaced with distilled water. Next, the pure water permeability of the above hollow fiber porous membranes was evaluated and found to be 3.9×10⁻⁹ m³/m²/s/Pa. The measurement of the permeability was performed at a head height of 1 m using purified water at 25° C. purified through a reverse osmosis membrane. The separation membrane module 2 was stored with its inside filled with water.

### Reference Example 3

### Leakage Test

Air at 100 kPa was supplied to the primary side of the separation membrane module 2 produced according to Reference Example 2. After water on the primary side of the separation membrane module 2 was filtered to the secondary side, the primary side of the separation membrane module 2 was isolated so as to be pressurized by the air at 100 kPa. A pressure gauge was provided in a supply line on the primary side of the separation membrane module 2 so that the pressure on the primary side of the separation membrane module 2 could be checked. The secondary side of the separation membrane module 2 was opened to the air. If a reduction in the pressure on the primary side of the separation membrane module 2 after 3 minutes was 10 kPa or less, the separation membrane module 2 was judged as pass.

### Example 1

The separation membrane module produced as described above was disposed in a fermented liquid circulation line of the membrane separation-type continuous fermentation apparatus 200A shown in FIG. 7, and sterilization was performed. First, 15 L of water was added to the fermenter 1, and the water was circulated from the fermenter 1 to the circulation pump 8 and then the separation membrane module 2 by the circulation pump 8. Then the filtration valve 13 of the separation membrane module 2 was opened. The filtration pump 11 was operated to perform filtration, whereby water was thereby sealed on the secondary side of the separation membrane module 2. The filtration was performed at a filtration flux of 0.2 m/day for 30 minutes. When water was sealed, the filtration valve 13, the washing solution valve 14, and the discharge valve 27 were closed. The amount of the sealed water was measured in advance, and it was found that the water was sealed in 98% of the secondary side volume of filtration portions of the hollow fiber membranes. After the water was sealed on the secondary side of the separation membranes, water in the fermenter 1, the circulation line, and the primary side of the separation membrane module 2 was discharged. Then saturated water vapor controlled at 125° C. was supplied from the vapor supply unit 20 to the fermenter 1. After the fermenter 1 reached 121° C., the water vapor was supplied to the pipes 23 and 25, the circulation pump 8, the separation membrane module 2, etc. A thermocouple was placed at the central portion of the bundle of the hollow fiber membranes of the separation membrane module 2 to observe the temperature inside the separation membrane module 2. The water vapor was supplied until the temperature of the thermocouple reached 123° C. to heat the separation membrane module 2. After the separation membrane module 2 was held at 123° C. or higher for 20 minutes, the supply of the water vapor was stopped, and the steam sterilization was terminated. After termination of the steam sterilization, the gas supply valve 30 was opened when the temperature of the fermenter 1 was reduced to 100° C. to supply compressed air to the separation membrane module 2 so that negative pressure was not generated in the separation membrane module 2, and the separation membrane module 2 was left to cool. Then the water sealed on the secondary side of the separation membrane module 2 was discharged, and the sterilization treatment of the separation membrane module 2 was thereby completed. The total time of supply of water vapor to the separation membrane module 2 was 32 minutes. The steam sterilization of the separation membrane module 2 was repeated, and no problem was found in the leakage test in Reference Example 3 until the number of repetitions of the steam sterilization treatment was 10. The pure water permeability of the hollow fiber membrane module after the steam sterilization treatment was repeated 10 times was 98% of the pure water permeability of the as-produced hollow fibers.

Continuous fermentation was performed using the membrane separation-type continuous fermentation apparatus 200A using the separation membrane module 2 subjected to steam sterilization. The operating conditions in Example 1 were as follows, unless otherwise specified.

Volume of fermenter 1: 20 (L)

Effective volume of fermenter 1: 15 (L)

Temperature setting of fermenter 1: 37 (° C.)

Amount of airflow to fermenter 1: nitrogen gas 2 (L/min)

Stirring rate in fermenter 1: 600 (rpm)

pH setting in fermenter 1: pH was adjusted to 6 with 3N Ca(OH)₂

Supply of lactic acid fermentation medium: A lactic acid fermentation medium was added such that the liquid volume in the fermenter 1 was constant at about 15 L

Amount of liquid circulated by fermented liquid circulation unit: 10 (L/min)

Control of flow rate through filtration membranes: Flow rate was controlled by a suction pump

Intermittent filtration treatment: Operation cycle including filtration treatment (9 minutes) and no filtration treatment (1 minute)

Membrane filtration flux: Variable within the range of 0.1 (m/day) or more and 0.3 (m/day) or less such that the transmembrane pressure difference was 20 kPa or less. When the transmembrane pressure difference increased continuously beyond this range, the continuous fermentation was terminated.

The culture medium used had been subjected to steam sterilization using saturated water vapor at 121° C. for 20 minutes. The microorganism used was Sporolactobacillus laevolacticus JCM2513 (SL strain), and the culture medium used was a lactic acid fermentation medium having a composition shown in Table 1. The concentration of lactic acid, i.e., a product, was evaluated by HPLC shown below under the following conditions.

TABLE 1
Lactic acid fermentation medium

| Component | Amount | |
|---|---|---|
| Glucose | 100 | g |
| Yeast Nitrogen base W/O amino acid | 6.7 | g |
| (Difco Laboratories, Inc.) | | |
| 19 types of standard amino acids | 152 | mg |
| excluding leucine | | |
| Leucine | 760 | mg |
| Inositol | 152 | mg |
| p-aminobenzoic acid | 16 | mg |
| Adenine | 40 | mg |
| Uracil | 152 | mg |
| Water | 892 | g |

Column: Shim-Pack SPR-H (manufactured by Shimadzu Corporation)

Mobile phase: 5 mM p-toluenesulfonic acid (0.8 mL/min)

Reaction phase: 5 mM p-toluenesulfonic acid, 20 mM bis-tris, 0.1 mM EDTA.2Na (0.8 mL/min)

Detection method: Electric conductivity

Column temperature: 45° C.

The optical purity of lactic acid was analyzed under the following conditions.

Column: TSK-gel Enantio L1 (manufactured by TOSOH Corporation)

Mobile phase: 1 mM aqueous copper sulfate solution

Flow rate: 1.0 mL/minute

Detection method: UV 254 nm

Temperature: 30° C.

The optical purity of L-lactic acid is calculated using the following Formula (1).

Optical purity(%)=100×(L−D)/(D+L) (1)

The optical purity of D-lactic acid is calculated using the following Formula (2).

Optical purity(%)=100×(D−L)/(D+L) (2)

Here, L represents the concentration of L-lactic acid, and D represents the concentration of D-lactic acid.

In the cultivation, the SL strain was first subjected to shake culture overnight in 5 mL of a lactic acid fermentation medium in a test tube (first preculture). The obtained culture solution was inoculated into 100 mL of a fresh lactic acid fermentation medium and subjected to shake culture in a 1000-mL Sakaguchi flask at 30° C. for 24 hours (second preculture). The second preculture solution was inoculated into a culture medium placed in the 15-L fermenter 1 of the continuous fermentation apparatus 200A shown in FIG. 7, and the fermenter 1 was stirred by the stirrer 4 equipped in the fermenter 1. The amount of airflow, temperature, and pH in the fermenter 1 were controlled, and cultivation was performed for 24 hours without actuation of the circulation pump 8 (final preculture). Immediately after completion of the final preculture, the circulation pump 8 was actuated. In this case, in addition to the operating conditions used during the final preculture, the lactic acid fermentation medium was continuously supplied. Continuous cultivation was performed while the amount of water passing through the membranes was controlled such that the amount of the fermented liquid in the continuous fermentation apparatus was 15 L, whereby D-lactic acid was produced by continuous fermentation. The amount of water passing through the membranes during a continuous fermentation test was controlled by the filtration pump 11 such that the filtration amount was the same as the amount of the fermentation medium supplied. The concentration of produced D-lactic acid in the fermented liquid that had passed through the membranes and the concentration of remaining glucose were measured as appropriate. Intermittent filtration treatment, i.e., cyclic operation including filtration treatment (9 minutes) and no filtration treatment (1 minute), was performed. The chemical was produced in the membrane separation-type continuous fermentation apparatus 200A shown in FIG. 7, and continuous fermentation could be performed for 400 hours.

### Example 2

The separation membrane module 2 was connected to the membrane separation-type continuous fermentation apparatus 200A shown in FIG. 7, and steam sterilization was performed in the same manner as in Example 1. First, 10 L of water was added to the fermenter 1, and the water was circulated through the fermenter 1, the circulation pump 8, and the separation membrane module 2 by the circulation pump 8. Then the filtration valve 13 of the separation membrane module 2 was opened. The filtration pump 11 was operated to perform filtration, whereby water was filtered to the secondary side of the separation membranes and sealed on the secondary side. The filtration was performed at a filtration flux of 0.1 m/day for 5 minutes. When water was sealed on the secondary side, the filtration valve 13, the washing solution valve 14, and the discharge valve 27 were closed. Then water in the fermenter 1 and the circulation line, and on the primary side of the separation membrane module 2 was discharged. The amount of the sealed water was measured in advance, and it was found that the water was sealed in 80% of the secondary side volume of the filtration portions of the hollow fiber membranes. Then saturated water vapor controlled at 125° C. was supplied from the vapor supply unit 20 to the fermenter 1. After the fermenter 1 reached 121° C., the water vapor was supplied to the pipes 23 and 25, the circulation pump 8, the separation membrane module 2, etc. A thermocouple was placed at the central portion of the bundle of the hollow fiber membranes of the separation membrane module 2 to observe the temperature inside the separation membrane module 2. The water vapor was supplied until the temperature of the thermocouple reached 123° C. to heat the separation membrane module 2. After the separation membrane module 2 was held at 123° C. or higher for 20 minutes, the supply of the water vapor was stopped, and the steam sterilization was terminated. After termination of the steam sterilization, the gas supply valve 30 was opened when the temperature of the fermenter 1 was reduced to 100° C. to supply compressed air to the separation membrane module 2 so that negative pressure was not generated in the separation membrane module 2, and the separation membrane module 2 was left to cool. Then the water sealed on the secondary side of the separation membrane module 2 was discharged, and the sterilization treatment of the separation membrane module 2 was completed. The total time of supply of water vapor to the separation membrane module 2 was 35 minutes. The steam sterilization of the separation membrane module 2 was repeated, and no problem was found in the leakage test in Reference Example 3 until the number of repetitions of the steam sterilization treatment was 10. The pure water permeability of the hollow fiber membrane module after the steam sterilization treatment was repeated 10 times was 99% of the pure water permeability of the as-produced hollow fibers.

### Example 3

The separation membrane module was connected to the membrane separation-type continuous fermentation apparatus 200A shown in FIG. 7, and steam sterilization was performed in the same manner as in Example 1. First, 10 L of a 10 wt % aqueous glycerin solution was added to the fermenter 1, and the aqueous glycerin solution was circulated between the fermenter 1 and the separation membrane module 2 by the circulation pump 8. Then the filtration valve 13 of the separation membrane module 2 was opened. The filtration pump 11 was operated to filtrate the aqueous glycerin solution to the secondary side of the separation membranes, whereby the aqueous glycerin solution was sealed on the secondary side of the separation membranes. The filtration was performed at a filtration flux of 0.2 m/day for 15 minutes. When the aqueous glycerin solution was sealed, the filtration valve 13, the washing solution valve 14, and the discharge valve 27 were closed. Then the aqueous glycerin solution in the fermenter 1 and the circulation line, and on the primary side of the separation membrane module 2 was discharged. The amount of the sealed aqueous glycerin solution was measured in advance, and it was found that the aqueous glycerin solution was sealed in 95% of the secondary side volume of the filtration portions of the hollow fiber membranes.

Then saturated water vapor controlled at 125° C. was supplied from the vapor supply unit 20 to the fermenter 1. After the fermenter 1 reached 121° C., the water vapor was supplied to the circulation line, the circulation pump 8, the separation membrane module 2, etc. A thermocouple was placed at the central portion of the bundle of the hollow fiber membranes of the separation membrane module 2 to observe the temperature inside the separation membrane module 2. The water vapor was supplied until the temperature of the thermocouple reached 123° C. to heat the separation membrane module 2. After the separation membrane module 2 was held at 123° C. or higher for 20 minutes, the supply of the water vapor was stopped, and the steam sterilization was terminated. The total time of supply of water vapor to the separation membrane module 2 was 40 minutes. After the steam sterilization, the gas supply valve 30 was opened when the temperature of the fermenter 1 was reduced to 100° C. to supply compressed air to the separation membrane module 2 so that negative pressure was not generated in the separation membrane module 2, and the separation membrane module 2 was left to cool. Then after the separation membrane module 2 was left to cool to 30° C., 1 L of a 30 wt % aqueous ethanol solution was added to the fermenter 1, and the aqueous ethanol solution was circulated between the fermenter 1 and the separation membrane module 2 by the circulation pump 8. Then the filtration valve 13 of the separation membrane module 2 was opened. The filtration pump 11 was operated to perform filtration, whereby the aqueous ethanol solution was filtered to the secondary side of the separation membranes. The filtration was performed at a filtration flux of 0.2 m/day for 15 minutes. Then the aqueous ethanol solution in the fermenter 1 and the circulation line, and on the primary and secondary sides of the separation membrane module 2 was discharged.

Then 10 L of water was added to the fermenter 1 and circulated between the fermenter 1 and the separation membrane module 2 by the circulation pump 8. Then the filtration valve 13 of the separation membrane module 2 was opened. The filtration pump 11 was operated to perform filtration, whereby the water was filtered to the secondary side of the separation membranes. The filtration was performed at a filtration flux of 0.2 m/day for 15 minutes. Then water in the fermenter 1 and the circulation line, and on the primary and secondary sides of the separation membrane module 2 was discharged, and the washing of the separation membranes was completed.

The steam sterilization of the separation membrane module 2 was repeated, and no problem was found in the leakage test in Reference Example 3 until the number of repetitions of the steam sterilization treatment was 10. The pure water permeability of the hollow fiber membrane module after the steam sterilization treatment was repeated 10 times was 99% of the pure water permeability of the as-produced hollow fibers.

### Example 4

The separation membrane module was connected to the membrane separation-type continuous fermentation apparatus 200A shown in FIG. 7, and steam sterilization was performed in the same manner as in Example 1. First, 10 L of water was added to the fermenter 1, and the water was circulated between the fermenter 1 and the separation membrane module 2 by the circulation pump 8. Then the filtration valve 13 of the separation membrane module 2 was opened. The filtration pump 11 was operated to filtrate the water to the secondary side of the separation membranes, whereby the water was sealed on the secondary side of the separation membranes. The filtration was performed at a filtration flux of 0.1 m/day for 5 minutes. The water was sealed by closing the filtration valve 13, the washing solution valve 14, and the discharge valve 27. Then water in the fermenter 1 and the circulation line, and on the primary side of the separation membrane module 2 was discharged. The amount of the sealed water was measured in advance, and it was found that the water was sealed in 85% of the secondary side volume of the filtration portions of the hollow fiber membranes.

Then saturated water vapor controlled at 125° C. was supplied from the vapor supply unit 20 to the fermenter 1. After the fermenter 1 reached 121° C., the water vapor was supplied to the circulation line, the circulation pump 8, the separation membrane module 2, etc. A thermocouple was placed at the central portion of the bundle of the hollow fiber membranes of the separation membrane module 2 to observe the temperature inside the separation membrane module 2. The water vapor was supplied until the temperature of the thermocouple reached 123° C. to heat the separation membrane module 2. After the separation membrane module 2 was held at 123° C. or higher for 20 minutes, the supply of the water vapor was stopped, and the steam sterilization of the separation membrane module 2 was terminated. After termination of the steam sterilization, the separation membrane module 2 was left to cool until its surface temperature became 100° C.

Then the separation membrane washing unit 18 was used to cool the separation membrane module. Specifically, the washing solution valve 14 was opened, and the washing solution supply pump 12 was actuated. Warm water at 80° C. was thereby passed from the secondary side of the separation membrane module to the primary side at a counter pressure filtration flux of 1 m/d for 5 minutes to cool the separation membrane module. After the separation membrane module 2 was cooled, the water sealed on the secondary side was discharged, and the sterilization treatment of the separation membrane module 2 was completed. The total time of supply of water vapor to the separation membrane module 2 was 35 minutes. The steam sterilization of the separation membrane module 2 was repeated, and no problem was found in the leakage test in Reference Example 3 until the number of repetitions of the steam sterilization treatment was 10. The pure water permeability of the hollow fiber membrane module after the steam sterilization treatment was repeated 10 times was 98% of the pure water permeability of the as-produced hollow fibers.

### Example 5

The separation membrane module with the liquid supply unit 40 connected to the secondary side (see FIG. 3) was connected to the membrane separation-type continuous fermentation apparatus, and steam sterilization was performed. First, the sealed liquid supply valve 22 was opened, and a 10 wt % aqueous glycerin solution was supplied from the liquid supply unit 40 to the secondary side of the separation membrane module 2 by the liquid supply pump 21. Then the aqueous glycerin solution was subjected to counter pressure filtration from the secondary side of the separation membrane module 2 to the primary side, and the aqueous glycerin solution was thereby sealed on the secondary side of the separation membranes. The counter pressure filtration was performed at a filtration flux of 0.1 m/day for 5 minutes. When the aqueous glycerin solution was sealed, the filtration valve 13, the washing solution valve 14, and the discharge valve 27 were closed. Then the aqueous glycerin solution subjected to counter pressure filtration to the primary side of the separation membrane module 2 was discharged. The amount of the sealed aqueous glycerin solution was measured in advance, and it was found that the aqueous glycerin solution was sealed in 85% of the secondary side volume of the filtration portions of the hollow fiber membranes.

Then saturated water vapor controlled at 125° C. was supplied from the vapor supply unit 20 to the fermenter 1. After the fermenter 1 reached 121° C., the water vapor was supplied to the circulation line, the circulation pump 8, the separation membrane module 2, etc. A thermocouple was placed at the central portion of the bundle of the hollow fiber membranes of the separation membrane module 2 to observe the temperature inside the separation membrane module 2. The water vapor was supplied until the temperature of the thermocouple reached 123° C. to heat the separation membrane module 2. After the separation membrane module 2 was held at 123° C. or higher for 20 minutes, the supply of the water vapor was stopped, and the steam sterilization of the separation membrane module 2 was terminated. The total time of supply of water vapor to the separation membrane module 2 was 35 minutes.

After the steam sterilization, the gas supply valve 30 was opened when the temperature of the fermenter was reduced to 100° C. to supply compressed air to the separation membrane module 2 so that negative pressure was not generated in the separation membrane module 2, and the separation membrane module 2 was left to cool. Then after the separation membrane module 2 was left to cool to 30° C., 1 L of a 30 mass % aqueous ethanol solution was added to the fermenter 1, and the aqueous ethanol solution was circulated between the fermenter 1 and the separation membrane module 2 by the circulation pump 8. Then the filtration valve 13 of the separation membrane module 2 was opened. The filtration pump 11 was operated to perform filtration, whereby the aqueous ethanol solution was filtrated to the secondary side of the separation membranes. The filtration was performed at a filtration flux of 0.2 m/day for 15 minutes. Then the aqueous ethanol solution in the fermenter 1 and the circulation line, and on the primary and secondary sides of the separation membrane module 2 was discharged.

Then 10 L of water was added to the fermenter 1 and circulated between the fermenter 1 and the separation membrane module 2 by the circulation pump 8. Then the filtration valve 13 of the separation membrane module 2 was opened. The filtration pump 11 was operated to perform filtration, whereby the water was filtered to the secondary side of the separation membranes. The filtration was performed at a filtration flux of 0.2 m/day for 15 minutes. Then water in the fermenter 1 and the circulation line, and on the primary and secondary sides of the separation membrane module 2 was discharged, whereby the washing of the separation membranes was completed.

The steam sterilization of the separation membrane module 2 was repeated, and no problem was found in the leakage test in Reference Example 3 until the number of repetitions of the steam sterilization treatment was 10. The pure water permeability of the hollow fiber membrane module after the steam sterilization treatment was repeated 10 times was 98% of the pure water permeability of the as-produced hollow fibers.

### Example 6

The separation membrane module with the liquid supply unit 40 connected to the secondary side (see FIG. 3) was connected to the membrane separation-type continuous fermentation apparatus 200A shown in FIG. 7, and steam sterilization was performed in the same manner as in Example 1. First, 10 L of water was added to the fermenter 1, and the water was circulated between the fermenter 1 and the separation membrane module 2 by the circulation pump 8. Then the filtration valve 13 of the separation membrane module 2 was opened. The filtration pump 11 was operated to filtrate the water to the secondary side of the separation membranes, whereby the water was sealed on the secondary side of the separation membranes. The filtration was performed at a filtration flux of 0.1 m/day for 5 minutes. The water was sealed by closing the filtration valve 13, the washing solution valve 14, the sealed liquid supply valve 22, and the discharge valve 27. Then water in the fermenter 1 and the circulation line, and on the primary side of the separation membrane module 2 was discharged. The amount of the sealed water was measured in advance, and it was found that the water was sealed in 85% of the secondary side volume of the filtration portions of the hollow fiber membranes. Then the liquid supply valve 22 was opened, and water was supplied from the liquid supply unit to the separation membrane module 2 by the liquid supply pump 21 and then subjected to counter pressure filtration from the secondary side of the separation membrane module 2 to the primary side. The water was thereby supplied to the secondary side of the separation membranes. The filtration was performed continuously at a filtration flux of 0.02 m/day.

While the counter pressure filtration was performed in the manner described above, saturated water vapor controlled at 125° C. was supplied from the vapor supply unit 20 to the fermenter 1. After the fermenter 1 reached 121° C., the water vapor was supplied to the circulation line, the circulation pump 8, the separation membrane module 2, etc. A thermocouple was placed at the central portion of the bundle of the hollow fiber membranes of the separation membrane module 2 to observe the temperature inside the separation membrane module 2. The water vapor was supplied until the temperature of the thermocouple reached 123° C. to heat the separation membrane module 2. After the separation membrane module 2 was held at 123° C. or higher for 20 minutes, the supply of the water vapor was stopped, and the steam sterilization of the separation membrane module 2 was terminated. The total time of supply of water vapor to the separation membrane module 2 was 40 minutes.

After the steam sterilization, the gas supply valve 30 was opened when the temperature of the fermenter 1 was reduced to 100° C. to supply compressed air to the separation membrane module 2 so that negative pressure was not generated in the separation membrane module 2, and the separation membrane module 2 was left to cool. Then the water sealed on the secondary side of the separation membrane module 2 was discharged, and the sterilization treatment of the separation membrane module 2 was completed.

The steam sterilization of the separation membrane module 2 was repeated, and no problem was found in the leakage test in Reference Example 3 until the number of repetitions of the steam sterilization treatment was 10. The pure water permeability of the hollow fiber membrane module after the steam sterilization treatment was repeated 10 times was 99% of the pure water permeability of the as-produced hollow fibers.

### Example 7

The separation membrane module was connected to the membrane separation-type continuous fermentation apparatus 200A shown in FIG. 5, and steam sterilization was performed in the same manner as in Example 1.

First, 10 L of water was added to the fermenter 1, and the water was circulated between the fermenter 1 and the separation membrane module 2 by the circulation pump 8. Then the filtration valve 13 of the separation membrane module 2 was opened. The filtration pump 11 was operated to filtrate the water to the secondary side of the separation membranes, whereby the water was sealed on the secondary side of the separation membranes. The filtration was performed at a filtration flux of 0.1 m/day for 5 minutes. The water was sealed by closing the filtration valve 13, the washing solution valve 14, and the discharge valve 27. Then water in the fermenter 1 and the circulation line, and on the primary side of the separation membrane module 2 was discharged. The amount of the sealed water was measured in advance, and it was found that the water was sealed in 85% of the secondary side volume of the filtration portions of the hollow fiber membranes.

Next, the temperature of the water in the fermenter 1 was increased to 50° C. Then the filtration valve 13, the washing solution valve 14, the discharge valve 27, and the liquid supply valve 22 were closed, and the circulation valve 17 was opened. The circulation pump 8 was actuated in this state, and warm water at 50° C. was subjected to crossflow circulation to the separation membrane module 2. Five minutes after the start of the circulation, the temperature of the fermenter was increased to 80° C. at 1° C./minute while the crossflow circulation was performed. Then the warm water in the fermenter 1 and on the primary side of the separation membrane module 2, as well as in the pipe for the retentate was discharged to the outside of the system.

Then saturated water vapor controlled at 125° C. was supplied from the vapor supply unit 20 to the fermenter 1. After the fermenter 1 reached 121° C., the water vapor was supplied to the circulation line, the circulation pump 8, the separation membrane module 2, etc. A thermocouple was placed at the central portion of the bundle of the hollow fiber membranes of the separation membrane module 2 to observe the temperature inside the separation membrane module 2. The water vapor was supplied until the temperature of the thermocouple reached 123° C. to heat the separation membrane module 2. After the separation membrane module 2 was held at 123° C. or higher for 20 minutes, the supply of the water vapor was stopped, and the steam sterilization of the separation membrane module 2 was terminated. After termination of the steam sterilization, the separation membrane module 2 was left to cool until its surface temperature became 100° C. Then warm water at 80° C. was passed from the secondary side of the separation membrane module to the primary side at a counter pressure filtration flux of 1 m/d for 5 minutes to cool the separation membrane module 2. After the separation membrane module 2 was cooled, the water sealed on the secondary side was discharged, and the sterilization treatment of the separation membrane module 2 was completed. The total time of supply of water vapor to the separation membrane module 2 was 30 minutes. The steam sterilization of the separation membrane module 2 was repeated, and no problem was found in the leakage test in Reference Example 3 until the number of repetitions of the steam sterilization treatment was 10. The pure water permeability of the hollow fiber membrane module after the steam sterilization treatment was repeated 10 times was 98% of the pure water permeability of the as-produced hollow fibers.

### Comparative Example 1

The separation membrane module was connected to the membrane separation-type continuous fermentation apparatus 200A shown in FIG. 7, and steam sterilization was performed in the same manner as in Example 1. First, 1 L of water was added to the fermenter 1, and the water was circulated between the fermenter 1 and the separation membrane module 2 by the circulation pump 8. Then the filtration valve 13 of the separation membrane module 2 was opened. The filtration pump 11 was operated to perform filtration, whereby the water was sealed on the secondary side of the separation membranes. Then water in the fermenter 1 and the circulation line, and on the primary side of the separation membrane module 2 was discharged. The filtration valve 13 and the discharge valve 27 were opened, and the water on the secondary side of the separation membranes was discharged. The amount of the sealed water was measured in advance under the same conditions, and it was found that the water was sealed in 10% of the secondary side volume of filtration portions of the hollow fiber membranes. Then saturated water vapor controlled at 125° C. was supplied from the vapor supply unit 20 to the fermenter 1. After the fermenter 1 reached 121° C., the water vapor was supplied to the circulation line, the circulation pump 8, the separation membrane module 2, etc. A thermocouple was placed at the central portion of the bundle of the hollow fiber membranes of the separation membrane module 2 to observe the temperature inside the separation membrane module 2. The water vapor was supplied until the temperature of the thermocouple reached 123° C. to heat the separation membrane module 2. After the separation membrane module 2 was held at 123° C. or higher for 20 minutes, the supply of the water vapor was stopped, and the steam sterilization of the separation membrane module 2 was terminated. After termination of the steam sterilization, the gas supply valve 30 was opened when the temperature of the fermenter 1 was reduced to 100° C. to supply compressed air to the separation membrane module 2 so that negative pressure was not generated in the separation membrane module 2, and the separation membrane module 2 was left to cool. The total time of supply of water vapor to the separation membrane module 2 was 55 minutes. The steam sterilization of the separation membrane module 2 was repeated. After the fourth steam sterilization treatment, the separation membrane module 2 failed the leakage test in Reference Example 3. The pure water permeability of the hollow fiber membrane module after the third steam sterilization treatment was 90% of the pure water permeability of the as-produced hollow fibers.

### INDUSTRIAL APPLICABILITY

The method of sterilizing a separation membrane module, the method of producing a chemical by continuous fermentation, the separation membrane module sterilizing apparatus, and the membrane separation-type continuous fermentation apparatus according to the present invention are useful for the production of a chemical, i.e., a fermentation product by a microorganism etc.

### REFERENCE SIGNS LIST

1 fermenter
2 separation membrane module
3 temperature controller
4 stirrer
5 pH sensor-controller
6 level sensor-controller
7 differential pressure sensor-controller
8 circulation pump
9 feedstock supply pump
10 neutralizer supply pump
11 filtration pump
12 washing solution supply pump
13 filtration valve
14 washing solution valve
15 gas supply unit
16 water supply pump
17 circulation valve
18 separation membrane washing unit
19 supply valve
20 vapor supply unit
21 liquid supply pump
22 liquid supply valve
23, 25, 34 pipe
24 filtrate discharge line
26, 33 discharge line
27, 32 discharge valve
29 washing solution supply line
30 gas supply valve
31 liquid supply line
40 liquid supply unit
50 controller
100, 100A, 100B sterilizing apparatus
200, 200A membrane separation-type continuous fermentation apparatus

1. A method of sterilizing a separation membrane module using water vapor, the method comprising:
a liquid supplying step of supplying a liquid having a boiling point of 80° C. or higher at atmospheric pressure to a secondary side of the separation membrane module such that a filling ratio of the liquid in a space surrounded by a filtration portion of a separation membrane is 70% or more, the filtration portion being used for filtration;
a liquid sealing step of isolating the secondary side of the separation membrane module such that the filling ratio of the liquid supplied to the secondary side in the liquid supplying step is 70% or more; and
a sterilization step of sterilizing the separation membrane module by supplying water vapor to a primary side of the separation membrane module while the secondary side of the separation membrane module is isolated.

2. The method of sterilizing the separation membrane module according to claim 1, wherein
the liquid supplying step is performed before the sterilization step and includes passing the liquid through separation membrane from the primary side of the separation membrane module to the secondary side or supplying the liquid directly to the secondary side and then passing the liquid through the separation membrane from the secondary side to the primary side, and
the method of sterilizing further comprises, after the liquid sealing step and before the sterilization step, a discharging step of discharging the liquid on the primary side of the separation membrane module.

3. The method of sterilizing the separation membrane module according to claim 1, wherein the liquid supplied to the separation membrane module in the liquid supplying step is water.

4. The method of sterilizing the separation membrane module according to claim 1, further comprising, after the sterilization step, a cooling step of cooling the separation membrane module by discharging the liquid sealed on the secondary side of the separation membrane module and supplying, to the secondary side, a liquid that is the same as or different from the liquid supplied in the supplying step.

5. The method of sterilizing the separation membrane module according to claim 1, further comprising:
a discharging step of discharging the liquid sealed on the secondary side of the separation membrane module after the sterilization step; and
a cooling step of cooling the separation membrane module by supplying a washing solution to the secondary side of the separation membrane module and discharging the washing solution from the secondary side of the separation membrane module to rinse an interior on the secondary side and to cool the separation membrane module.

6. The method of sterilizing the separation membrane module according to claim 1, further comprising a pre-heating step of pre-heating the separation membrane module by supplying warm water thereinto before heating step.

7. The method of sterilizing the separation membrane module according to claim 1, wherein the sterilization step includes supplying water vapor to the primary side of the separation membrane while the liquid is passed from the secondary side of the separation membrane to the primary side.

8. A method of producing a chemical by continuous fermentation, the method comprising:
a steam sterilization step of using the method of sterilizing according to claim 1 to sterilize the separation membrane module;
a fermentation step of converting a fermentation feedstock to a fermented liquid containing a chemical by fermentation culture by a microorganism; and
a membrane separation step of collecting the chemical as a filtrate from the fermented liquid using the separation membrane module subjected to the steam sterilization step.

9. A membrane separation-type continuous fermentation apparatus, comprising:
a fermenter configured to convert a fermentation feedstock to a fermented liquid containing a chemical by fermentation cultivation of the fermentation feedstock using a microorganism;
a separation membrane module configured to separate the chemical from the fermented liquid;
a fermented liquid circulation unit configured to feed the fermented liquid from the fermenter to the separation membrane module;
a steam supply unit configured to supply water vapor to the fermenter and the separation membrane module;
a liquid supply unit configured to supply a liquid having a boiling point of 80° C. or higher at atmospheric pressure to a secondary side of the separation membrane module; and
an isolation unit configured to isolate the secondary side of the separation membrane module such that a filling ratio of the liquid in a space surrounded by a filtration portion of a separation membrane is 70% or more during operation of the stream supply unit, the filtration portion being on the secondary side of the separation membrane module and used for filtration.

10. The method of sterilizing the separation membrane module according to claim 2, wherein the liquid supplied to the separation membrane module in the liquid supplying step is water.

11. The method of sterilizing the separation membrane module according to claim 2, further comprising, after the sterilization step, a cooling step of cooling the separation membrane module by discharging the liquid sealed on the secondary side of the separation membrane module and supplying, to the secondary side, a liquid that is the same as or different from the liquid supplied in the supplying step.

12. The method of sterilizing the separation membrane module according to claim 3, further comprising, after the sterilization step, a cooling step of cooling the separation membrane module by discharging the liquid sealed on the secondary side of the separation membrane module and supplying, to the secondary side, a liquid that is the same as or different from the liquid supplied in the supplying step.

13. A method of producing a chemical by continuous fermentation, the method comprising:
a steam sterilization step of using the method of sterilizing according to claim 2 to sterilize the separation membrane module;
a fermentation step of converting a fermentation feedstock to a fermented liquid containing a chemical by fermentation culture by a microorganism; and
a membrane separation step of collecting the chemical as a filtrate from the fermented liquid using the separation membrane module subjected to the steam sterilization step.

14. A method of producing a chemical by continuous fermentation, the method comprising:
a steam sterilization step of using the method of sterilizing according to claim 3 to sterilize the separation membrane module;
a fermentation step of converting a fermentation feedstock to a fermented liquid containing a chemical by fermentation culture by a microorganism; and
a membrane separation step of collecting the chemical as a filtrate from the fermented liquid using the separation membrane module subjected to the steam sterilization step.

15. A method of producing a chemical by continuous fermentation, the method comprising:
a steam sterilization step of using the method of sterilizing according to claim 4 to sterilize the separation membrane module;
a fermentation step of converting a fermentation feedstock to a fermented liquid containing a chemical by fermentation culture by a microorganism; and
a membrane separation step of collecting the chemical as a filtrate from the fermented liquid using the separation membrane module subjected to the steam sterilization step.
